(19) United States
(12) Patent Application Publication
(10) Pub. No.: US 2008/0138635 A1
(43) Pub. Date: Jun. 12, 2008

(54) Conjugated Organic Molecules for Molecular Electronic Devices

(76) Inventors: Zhikuan Chen, Singapore (SG); Chun Huang, Singapore (SG); Jianshu Yang, Singapore (SG); Sean O'Shea, Singapore (SG); Kian Ping Loh, Singapore (SG)

Correspondence Address: KLARQUIST SPARKMAN, LLP, 121 SW SALMON STREET, SUITE 1600, PORTLAND, OR, 97204, US

(21) Appl. No.: 11/885,083

(22) PCT Filed: Mar. 2, 2005
(86) PCT No.: PCT/SG05/00066

§ 371 Date: Aug. 24, 2007

(51) Int. Cl. B32B 15/04; B05D 5/12; B32B 9/04; C07C 43/225; C07C 323/16; B32B 9/00

(52) U.S. Cl. 428/447; 428/704; 428/457; 427/77; 568/49; 568/643

(57) ABSTRACT

There is provided a conjugated molecule that is useful as a conductive path in an electronic device. The conjugated molecule includes at least one p/n junction so as to provide a direction to electron flow and one end alligator clip group which allows for self-orientation of the molecule during assembly in a device, resulting in an asymmetric structure of the molecule. The conjugated molecule may be used in diodes, molecular switches, transistors, and in the manufacture of memory devices.

### FIELD OF THE INVENTION

The present invention relates generally to molecules suitable for use in molecular electronic devices, and particularly to conjugated organic molecules.

### BACKGROUND OF THE INVENTION

Molecular electronics is a promising new technology for high-speed computation that uses a single molecule or a group of molecules to perform the basic functions of Si-based electronic devices to overcome size and fundamental physical problems of silicon-based technology. It is estimated that a typical 1 cm² Pentium™ chip can fit 10¹⁴ single molecule devices, while only 10⁷ to 10⁸ silicon-based devices can be packed in the same area. Hence, the density, which correlates to the operating speed of the chip, can technically be improved by at least a million times using molecular devices. In addition, molecule-based device construction is a bottom-up process, in which the synthesized functional molecules are further self-assembled into desired circuits. Therefore, it is a rapid and cost-effective technology.

To construct molecular electronic devices, molecular-scale materials are required to function as the active channels for charge or signal transport for different devices, for example, for molecular wires, diodes, switches, transistors, logic gates, memory devices and the like. Thus, an active area of research is the development of molecular-scale materials that mimic the traditional silicon-based devices.

The molecules used as the conductive material must be able to perform functions analogous to those of Si-based devices in microelectronics. A number of materials have been used for molecular scale devices, for example, carbon nanotubes, oxide nanotubes/wires (Lieber C. M. et al., “High performance silicon nanowire field effect transistors”, Nano. Lett., 2003, Vol. 3, 149-152), metal complexes (Rack J. J., U.S. Pat. No. 6,433,270), DNA (Fink H. W. et al., “Electrical conduction through DNA molecules”, Nature, 1999, Vol. 398, 407-410; Choo Y. et al., US 20030092010) and conjugated oligomers.

Carbon nanotubes have been used for field effect transistors and gain has been achieved by applying a voltage to a submerged gate beneath a single walled nanotube (Dekker C. et al, “Logic circuits with carbon nanotube transistors”, Science, 2001, Vol. 294, 1317-1320; “Room-temperature transistor based on a single carbon nanotube”, Nature, 1998, Vol. 393, 49-52). However, a transistor assembled in this way may or may not work, depending on whether the chosen nanotube is semiconducting or metallic. It's rather difficult to distinguish semiconducting tubes from metallic and insulating tubes. In addition, carbon nanotubes are not easily processed. Typically, an atomic force microscope is used to position the tubes on a substrate to attain the desired orientation. Oxide nanotubes or wires tend to have similar problems as carbon nanotubes.

DNA is another type of material that has potential for use in molecular electronics (Rakitin A, et al., Phys. Rev. Lett., 2001, Vol. 86: 3670-3673). The switching function performed by DNA-based devices depends on the DNA duplex association-dissociation, which is a rather slow process, which may limit the application of DNA-based devices in molecular electronics.

Organic molecules have been employed in molecular devices. Metzger and co-workers demonstrated a rectifying device based on multilayer of hexadecylquinolinium tricyanoquinodimethanide (“Unimolecular Electrical Rectification in Hexadecylquinolinium Tricyanoquinodimethanide”, J. Am. Chem. Soc. 1997, Vol. 119, 10455-10466). This molecule requires the use of Langmuir-Blodgett technique for deposition into films, which is not convenient for device fabrication. In addition, the adhesion of molecules to the electrode in this device is driven by physical adsorption, which is not a stable, reliable connection for monolayer or single molecule devices.

Rotaxanes and catenanes have also been used for fabrication of molecular logic gates by the group of Heath, Williams, and Stoddart (“Electronically configurable molecular-based logic gates”, Science, 1999, Vol. 285, page 391-394; Heath J. R. et al., U.S. Pat. No. 6,198,655). These molecules are not easily synthesized or easily integrated into molecular devices, and the response speed of the molecules to an electric field can be fairly slow.

U.S. Pat. No. 6,756,605, issued to Reed et al. describes molecular scale electronic devices which include conjugated organic molecules as the conductive path. The conductive molecules described include chains of aromatic molecules separated, for example, by a triply bonded ethynylene group, and containing at least one electron withdrawing substituent on the backbone of the conjugated organic molecule. The conductive path can be given the property of a resonant tunnelling diode by the inclusion of a non-conjugated spacer group through which electrons must tunnel in order to travel along the conductive path. These molecular scale devices are useful as wires, or as resonant tunnelling diodes which regulate current flow as a function of voltage.

Ellenbogen and Love further expand on the resonant tunnelling diodes developed by Reed and Tour, and suggest the addition of intramolecular dopants to either side of a spacer group in order to convert a tunnelling diode to a rectifier diode by having the portions of the molecule on either side of the spacer group with different energy levels (MP 98W0000183, “Architectures for molecular electronic computers: 1. Logic structures and an adder built from molecular electronic diodes”, July 1999, The Mitre Group: McLean, Va.). However, such molecules are not easily assembled into a device in the proper orientation for diode operation.

Not only must the molecular conductive materials function as charge semi-conductors, such materials should be suitable for manufacture into devices in a simple and economic way. As well, such molecules should be useful as diodes and switches to allow for construction of more complex higher order devices.

### SUMMARY OF THE INVENTION

The present invention relates to conjugated molecules that include at least one p/n junction so as to provide a direction to electron flow and one end alligator clip group which allows for self-orientation of the molecule during assembly in a device, resulting in an asymmetric structure of the molecules. The conjugated molecules may be used as diodes, molecular switches, transistors, and in the manufacture of memory devices.

In one aspect, there is provided a conjugated molecule comprising: from 3 to 100 Ar groups forming the backbone of the molecule, each Ar group being an arylene, an arylene-vinylene or an arylene-ethynylene group, at least one of the Ar groups being substituted with one or more electron-donating groups to form a p-type Ar group and at least one of the Ar groups being substituted with one or more electron-withdrawing groups to form an n-type Ar group, the p-type Ar group being adjacent to the n-type Ar group to form a p/n junction; and an AC group at one end of the backbone, the AC group capable of reacting with a conducting surface.

In another aspect, there is provided a molecular electronic device, comprising a first electrical contact, a second electrical contact, and the conjugated molecule described herein forming a conductive path between the first electrical contact and the second electrical contact, wherein the second electrical contact is connected to the conjugated molecule through the AC group.

In a further aspect, there is provided a method of manufacturing the molecular electronic described herein, comprising contacting the first electrical contact with a solution containing the conjugated molecule of claim 1 to form a monolayer of conjugated molecule on the first electrical contact, the first electrical contact contacting the AC group of the conjugated molecule; and depositing a second electrical contact on the monolayer of conjugated molecule, the second electrical contact contacting the end of the conjugated molecule not having the AC group.

In yet another aspect, there is provided a crossbar device comprising a first conductor and a second conductor that intersects the first conductor at a non-zero angle and the molecular electronic device described herein, wherein the molecular electronic device connects the first conductor and the second conductor at the point of intersection.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of example only, embodiments of the present invention,

FIG. 1 is a schematic diagram illustrating the preparation of 4-(2″, 5″-dimethoxyphenyl)-4′-(p-acetylthiophenyl)-2,2′,5,5′-tetramethoxy-1,1′-biphenyl;

FIG. 2 is a schematic diagram illustrating the preparation of a conjugated molecule, 4-(2′,5′-dimethoxy-4′-acetylthiophenyl)phenyl-nonafluorobiphenyl;

FIG. 3 is a schematic diagram illustrating the preparation of a conjugated molecule, 1-acetylthiophenyl-4-2′,2″,5′,5″-tetramethoxybiphenyl-tetrafluorobenzene;

FIG. 4 is a schematic diagram illustrating the preparation of 4′-acetylthio-biphenyl-4-yl-nonafluorobiphenyl-4-yl methane;

FIG. 5 is a schematic diagram illustrating the preparation of 4′-acetylthiobiphenyl-4-yl-nonafluorobiphenyl-4-yl ether;

FIG. 6 is a schematic diagram illustrating the preparation of 4′-acetylthiobiphenyl-4-yl-nonafluorobiphenyl-4-yl sulfide;

FIG. 7 is a graphical representation of the energy levels of the electron orbitals and the direction of electron flow across a conjugated molecule having a p/n junction;

FIG. 8 is a schematic diagram illustrating the assembly of a conjugated molecule 4-acetylthiophenyl-nonafluorobiphenyl assembled on a scanning tunnelling microscope tip;

FIG. 9 is a graph illustrating the I-V characteristics of 4-acetylthiophenyl-nonafluorobiphenyl;

FIG. 10 is a schematic illustration of a molecular electronic device comprising a conjugated molecule and two electrical contacts coupling to the conjugated molecule;

FIG. 11 is a cyclic voltammogram of 4-(p-tert-butylthiophenyl)-2,2′, 5,5′-tetramethoxybiphenyl (TSBOO), p-tert-butylthiophenyl-nonafluorobiphenyl (TSBFF) and tert-butylthiophenyl-4-(2′,5′-dimethoxyphenyl)-tetrafluorobenzene (TSBFO); and

FIG. 12 is a schematic illustration of a crossbar incorporating a molecular electronic device.

### DETAILED DESCRIPTION

In traditional silicon-based devices, p-type silicon can be placed adjacent to n-type silicon to create a p/n junction, and such junctions can be used to create a semiconductor device which will conduct in one direction only under normal operating conditions, for example, a diode. When a diode is properly assembled within a larger electronic device, current is regulated to only flow when the device is forward biased.

Similarly, the present devices are based on the creation of a p/n junction in a conductive organic molecule, which provides a direction to the conductance of the molecule. Such molecules are useful in the manufacture of devices such as diodes, switches and transistors, but require a mechanism for orienting the molecule when being assembled into an electronic device, in order that the molecule properly regulates current flow in the desired manner.

Thus, there is provided a conjugated molecule for use in constructing molecular electronic devices. The conjugated molecule is asymmetric and has at least two termini, each for coupling to an electronic connection, at least one p/n junction and an alligator clip group at one terminus for orienting the molecule when assembling in an electronic device. Generally, the conjugated molecule has from 3 to 100 arylene, arylene-vinylene or arylene-ethynylene groups forming the backbone of the molecule, with the alligator clip group situated at one end of the backbone. At least a first one of the arylene, arylene-vinylene or arylene-ethynylene groups is substituted with one or more electron-donating groups and at least a second one of the arylene, arylene-vinylene or arylene-ethynylene groups is substituted with one or more electron-withdrawing groups so as to form a p/n junction.

A p/n junction as used herein refers to the interface that occurs between adjacent electron-rich and electron-deficient segments of the backbone of the molecule created by substituting arylene, arylene-vinylene or arylene-ethynylene groups with electron-donating and electron-withdrawing groups, respectively. When an arylene, arylene-vinylene or arylene-ethynylene group that has a tendency to give up electrons is placed adjacent in the backbone to an arylene, arylene-vinylene or arylene-ethynylene group that has a tendency to accept electrons, the conductive path of the molecule is thus designed to conduct electrons in one direction only under normal operating conditions. Adjacent electron-rich and electron-deficient regions that form a p/n junction are positioned next to each other, and may be separated by a spacer groups as described below, resulting in a boundary between the conductive nature of the two regions which allows for conductance of electrons in one direction along the conjugated backbone of the molecule but not the other.

The term “conjugated” as used herein in reference to the backbone of a molecule refers to a molecule having two or more double and/or triple bonds in the main chain of the molecule, each double or triple bond being separated from the next consecutive double or triple bond by a single bond so that pi orbitals overlap not only across the double or triple bond, but also across adjacent single bonds located between adjacent double and/or triple bonds.

The conjugated molecule comprises arylene, arylene-vinylene and/or arylene-ethynylene groups, wherein at least two of the arylene, arylene-vinylene and/or arylene-ethynylene groups are substituted independently with one or more substituents, and together provide a p/n junction, as discussed below.

An “arylene group” as used herein is a bivalent radical derived from an aromatic compound. An aromatic compound is a cyclic compound having 4n+2 pi electrons where n is an integer equal to or greater than 0, and includes hydrocarbon aromatic compounds, for example benzene, and heteroaromatic compounds, for example pyridine. A “vinylene group” as used herein is the bivalent radical represented by the formula —CH═CH— and an “ethynylene group” as used herein is the bivalent radical represented by the formula —C≡C—. The symbol “Ar” as used herein refers generally to an arylene group, an arylene group and an adjacent vinylene group (“arylene-vinylene”) or an arylene group and an adjacent ethynylene group (“arylene-ethynylene”).

In one embodiment, the conjugated molecule has the general

[Ar]_{q}—[Ar^{p}]—[Arⁿ]—[Ar]ᵣ-AC Formula I

In the above Formula, each Ar group may be either an Ar^{p} or an Arⁿ group unless specified, and Ar, Ar^{p} and Arⁿ are each generically referred to as an Ar group. Thus, each Ar group is an arylene, an arylene-vinylene or an arylene-ethynylene group, and each Ar group may be the same or different, but an Ar^{p} group will have different a substituent or substituents from an Arⁿ group.

For example, each Ar group may independently be phenylene, naphthylene, thienylene, furylene, pyrrolylene, pyridylene, thiazolylene, oxadiazolylene, pyrazinylene, fluorenylene, indenofluorenylene, carbazolylene, indenocarbazolylene, dibenzofuranylene, dibenzothienylene, anthracenylene, tetracenylene, pentacenylene, indenylene, azulenylene, pentalenylene, heptalenylene, biphenylenylene, indacenylene, acenaphthenylene, phenalenylene, phenanthrylene, triphenylenylene, pyrenylene, naphthacenylene, hexacenylene, pyrazolylene, imidazolylene, naphthothienylene, thianthrenylene, pyranylene, isobenzofuranylene, chromenylene, xanthenylene, phenoxathiinylene, pyrimidinylene, pyridazinylene, indolizinylene, isoindolylene, indolylene, purinylene, quinolizinylene, quinolylene, phthalazinylene, pteridinylene, acridinylene, phenanthridinylene, pyrrolinylene, imidazolinylene, indolinylene, phenylene vinylene, naphthylene vinylene, thienylene vinylene, furylene vinylene, pyrrolylene vinylene, pyridylene vinylene, thiazolylene vinylene, oxadiazolylene vinylene, pyrazinylene vinylene, fluorenylene vinylene, indenofluorenylene vinylene, carbazolylene vinylene, indenocarbazolylene vinylene, dibenzofuranylene vinylene, dibenzothienylene vinylene, anthracenylene vinylene, tetracenylene vinylene, pentacenylene vinylene, indenylene vinylene, azulenylene vinylene, pentalenylene vinylene, heptalenylene vinylene, biphenylenylene vinylene, indacenylene vinylene, acenaphthenylene vinylene, phenalenylene vinylene, phenanthrylene vinylene, triphenylenylene vinylene, pyrenylene vinylene, naphthacenylene vinylene, hexacenylene vinylene, pyrazolylene vinylene, imidazolylene vinylene, naphthothienylene vinylene, thianthrenylene vinylene, pyranylene vinylene, isobenzofuranylene vinylene, chromenylene vinylene, xanthenylene vinylene, phenoxathiinylene vinylene, pyrimidinylene vinylene, pyridazinylene vinylene, indolizinylene vinylene, isoindolylene vinylene, indolylene vinylene, purinylene vinylene, quinolizinylene vinylene, quinolylene vinylene, phthalazinylene vinylene, pteridinylene vinylene, acridinylene vinylene, phenanthridinylene vinylene, pyrrolinylene vinylene, imidazolinylene vinylene, indolinylene vinylene, phenylene ethynylene, naphthylene ethynylene, thienylene ethynylene, furylene ethynylene, pyrrolylene ethynylene, pyridylene ethynylene, thiazolylene ethynylene, oxadiazolylene ethynylene, pyrazinylene ethynylene, fluorenylene ethynylene, indenofluorenylene ethynylene, carbazolylene ethynylene, indenocarbazolylene ethynylene, dibenzofuranylene ethynylene, dibenzothienylene ethynylene, anthracenylene ethynylene, tetracenylene ethynylene, pentacenylene ethynylene, indenylene ethynylene, azulenylene ethynylene, pentalenylene ethynylene, heptalenylene ethynylene, biphenylenylene ethynylene, indacenylene ethynylene, acenaphthenylene ethynylene, phenalenylene ethynylene, phenanthrylene ethynylene, triphenylenylene ethynylene, pyrenylene ethynylene, naphthacenylene ethynylene, hexacenylene ethynylene, pyrazolylene ethynylene, imidazolylene ethynylene, naphthothienylene ethynylene, thianthrenylene ethynylene, pyranylene ethynylene, isobenzofuranylene ethynylene, chromenylene ethynylene, xanthenylene ethynylene, phenoxathiinylene ethynylene, pyrimidinylene ethynylene, pyridazinylene ethynylene, indolizinylene ethynylene, isoindolylene ethynylene, indolylene ethynylene, purinylene ethynylene, quinolizinylene ethynylene, quinolylene ethynylene, phthalazinylene ethynylene, pteridinylene ethynylene, acridinylene ethynylene, phenanthridinylene ethynylene, pyrrolinylene ethynylene, imidazolinylene ethynylene or indolinylene ethynylene.

An Ar group may be substituted on the arylene portion of the Ar group with one or more substituents independently selected from the group consisting of linear or branched C₁₋₁₈ alkyl, linear or branched C₂₋₁₈ alkenyl, linear or branched C₂₋₁₈ alkynyl, linear or branched C₁₋₁₈ alkoxy, linear or branched C₁₋₁₈ alkylamino or dialkylamino, linear or branched C₁₋₁₈ alkylthio, amido, carbonyl, carboxyl, alkyl sulfonyl, sulfo, sulfonyl, thioamide, ^{]}C₅₋₃₀ aryl, C₅₋₃₀ arylamino, C₅₋₃₀ diarylamino, amino, ammonio, hydroxyl, nitro, cyano, isocyano and halide, wherein any of the C₁₋₁₈ alkyl, C₂₋₁₈ alkenyl, C₂₋₁₈ alkynyl, C₁₋₁₈ alkoxy, C₁₋₁₈ alkylamino or dialkylamino, or C₁₋₁₈ alkylthio may optionally contain one to four heteroatoms selected from the group consisting of N, O, S, Si and P, and wherein any of the C₁₋₁₈ alkyl, C₂₋₁₈ alkenyl, C₂₋₁₈ alkynyl, C₁₋₁₈ alkoxy, C₁₋₁₈ alkylamino or dialkylamino, C₁₋₁₈ alkylthio, C₅₋₃₀ aryl, C₅₋₃₀ arylamino or C₅₋₃₀ diarylamino may optionally be further substituted, including with one or more halides.

Furthermore, where any one of the Ar groups is an arylene-vinylene group, such an Ar group may be substituted on the vinylene moiety of the arylene-vinylene with one or two substituents independently selected from the group consisting of linear or branched C₁₋₁₈ alkyl, linear or branched C₂₋₁₈ alkenyl, linear or branched C₂₋₁₈ alkynyl, linear or branched C₁₋₁₈ alkoxy, linear or branched C₁₋₁₈ alkylamino or dialkylamino, linear or branched C₁₋₁₈ alkylthio, amido, carbonyl, carboxyl, alkyl sulfonyl, sulfo, sulfonyl, thioamide, ^{]}C₅₋₃₀ aryl, C₅₋₃₀ arylamino, C₅₋₃₀ diarylamino, amino, ammonio, hydroxyl, nitro, cyano, isocyano and halide, wherein any of the C₁₋₁₈ alkyl, C₂₋₁₈ alkenyl, C₂₋₁₈ alkynyl, C₁₋₁₈ alkoxy, C₁₋₁₈ alkylamino or dialkylamino, or C₁₋₁₈ alkylthio may optionally contain one to four heteroatoms selected from the group consisting of N, O, S, Si and P, and wherein any of the C₁₋₁₈ alkyl, C₂₋₁₈ alkenyl, C₂₋₁₈ alkynyl, C₁₋₁₈ alkoxy, C₁₋₁₈ alkylamino or dialkylamino, C₁₋₁₈ alkylthio, C₅₋₃₀ aryl, C₅₋₃₀ arylamino or C₅₋₃₀ diarylamino may optionally be further substituted, including with one or more halides.

Thus, any one of the Ar groups may be substituted as described above, where at least two Ar groups are substituted to form a p/n junction. The maximum number of substituents on any one of the Ar groups, either on the arylene moiety or where applicable on the vinylene moiety, is determined by the number of sites available on the particular Ar group for substitution. A skilled person will understand that any site on the backbone that is occupied by a hydrogen atom may potentially be substituted. As will be further understood, certain substituents, such as large, bulky substituents, may not be substituted at certain sites on the backbone where the arrangement of the backbone atoms or other substituents sterically hinders the substitution at those sites by particular groups.

The conductive nature of the conjugated molecule is modulated through the placement of one or more substituents on the Ar groups of the molecule to create the Ar^{p} and Arⁿ groups, to create at least one p/n junction.

An Ar^{p} group is an electron-rich Ar group, or is substituted with one or more electron-donating groups so as to have a tendency to donate electrons to an adjacent Ar groups along the backbone, rendering the Ar group a p-type Ar group. One or more electron-donating groups attached to an Ar group of the backbone of the conjugated molecule, so as to create an electron-donating Ar group or a p-type Ar group, provides that portion of the molecule with the nature of a p-type conductor. As will be apparent to a skilled person, electron-donating groups have electron-rich atoms or groups adjacent to the backbone of the conjugated molecule so as to push additional electrical charge into the conjugated system.

For example, electron-donating groups include alkoxyl, alkylthio, amino, hydroxyl, amido connected to the backbone through the nitrogen, carboxyl connected to the backbone through the oxygen, phenyl, naphthyl, thienyl, furyl, pyrrolyl, carbazolyl, alkyl, alkenyl and alkynyl.

Additionally, certain unsubstituted Ar groups will be Ar^{p} groups, and will be electron-rich, for example phenylene, naphthylene, thienylene, furylene and pyrrolylene. Substitution of an electron-rich Ar group with an electron-withdrawing substituent may convert the Ar group to an electron-deficient Ar group. An Arⁿ group is an electron-deficient Ar group or an Ar group substituted with one or more electron withdrawing groups so as to have a tendency to accept electrons from adjacent Ar groups along the backbone, rendering the Ar group an n-type Ar group. One or more electron-withdrawing groups attached to a backbone Ar group provide the conjugated molecule with the nature of an electron-deficient n-type conductor. The ability of such an Ar group (an electron-withdrawing Ar group or an n-type Ar group) to withdraw electrons from a neighbouring group tends to make an electron-withdrawing Ar group more electron-dense than a neighbouring Ar group that is not electron-withdrawing, similar to n-type materials used in a Si semiconductor. A skilled person will understand which substituents are electron-withdrawing. Generally, electron-withdrawing groups are groups that create a positive or delta-positive region adjacent to the backbone so as to pull electrons from the backbone toward the substituent.

For example, electron-withdrawing groups include halide, carbonyl, carboxyl, cyano, ammonio, nitro, sulfonyl, amido linked to the backbone through the oxygen, pyridinium, phosphonium, pyridyl, thiazolyl, oxadiazolyl and triazolyl groups.

As well, certain Ar groups, when unsubstituted, will be electron-deficient. For example, pyridylene, thiazolylene, oxadiazolylene and triazolylene are electron-deficient Ar groups. However, substitution of an unsubstituted electron-withdrawing Ar group with an electron-donating substituent may convert the Ar group to an electron-donating Ar, as will be appreciated by a skilled person.

A p/n junction is the interface between adjacent p-type Ar and n-type Ar groups, and imparts a conductive direction to the backbone of the molecule. In the Formula I, the junction occurs between the depicted Ar^{p} and Arⁿ groups, which may occur at any point along the backbone where an Ar^{p} group is adjacent to an Arⁿ group. The conjugated molecule contains at least one p/n junction.

The arrangement of Ar groups and substituents on the conjugated backbone may be selected to result in certain segments of the backbone being composed of Ar^{p} groups (a p-segment) or Arⁿ groups (an n-segment). Generally, a segment is a section of the molecule composed of one or more consecutive Ar groups that is of the same electron-rich or electron-deficient character, or that each has substituents of the same electron-donating or electron-withdrawing character.

It will be appreciated that Formula I set out above is one example of how the p-segments and n-segments may be arranged along the backbone of the conjugated molecule, and that the molecules described herein are not so limited. Rather, Ar groups, including substituents on the Ar groups, may be chosen so as to have consecutive blocks of p-segment and n-segment in the conductive path along the backbone.

For example, the molecule may have an n-segment followed by a p-segment followed by an AC group.

It will also be appreciated that although the above description has been directed to a two terminal molecule, the present molecules may be designed to have three or more terminals by designing a branch point at an Ar group within the chain, creating a conjugated molecule with a branched backbone. Thus, various molecules having multiple p/n junctions may be created. For example, a p-n-p type molecule, which will have two p/n junctions, may be created by having a segment of electron-deficient Arⁿ groups sandwiched between two segments of electron-rich Ar^{p} groups. By creating a branch point within the n segment, such a molecule can be used as a conductive path in a three terminal device, for example a transistor.

Similarly, an n-p-n type molecule, which will also have two p/n junctions, may be created by having a segment of electron-rich Ar^{p} groups sandwiched between two segments of electron-deficient Arⁿ groups.

The backbone may also be designed to have alternating p-n or n-p segments by having repeating segments of alternating electron-rich Ar^{p} groups and electron-deficient Arⁿ groups.

The conjugated molecule contains at least three Ar groups, but may, for example, contain up to 100 of such groups, each of which may be the same or different from any other Ar group in the molecule. Thus, q and r in Formula I are each integers from 0 to 98, and together q+r is from 1 to 98, resulting in from 3 to 100 total Ar groups, including the depicted Ar^{p} and Arⁿ groups that together form the at least one p/n junction. Preferably q and r are chosen so that q+r is from 1 to 18, resulting in from 3 to 20 total Ar groups in the conjugated molecule.

The conjugated molecule is asymmetric in that it contains an AC group at one end of the molecule. The AC group acts as an alligator clip group. As will be understood by a skilled person, the term “alligator clip” or “alligator clip group”, as used herein in reference to an end group within a conjugated molecule, refers to a group that reacts with a particular surface so as to form a covalent bond between the molecule containing the alligator clip group and the surface.

The AC group may be conjugated or non-conjugated with the backbone, and may be selected from acetylthio, methylthio, tert-butylthio, benzylthio, isocyano, diazo, phosphate, phosphonio, and phosphonitryl, or a derivative of any of such groups, any of which acetylthio, methylthio, tert-butylthio, benzylthio, phosphate or phosphonio may be substituted with C₁₋₁₈ alkyl, C₁₋₁₈ alkenyl, C₁₋₁₈ alkynyl, amido, carbonyl, sulfonyl, thioamide, C₅₋₃₀ aryl, C₅₋₃₀ arylamino, amino, nitro, cyano, isocyano and halide.

Preferably, the Ar group immediately adjacent to the AC group does not result in a vinylene or an ethynylene moiety being positioned adjacent to the AC group, since such positioning may result in an unstable structure, depending on the AC group that is used. For example, an AC group containing a thiol group or a derivative thereof should not be placed adjacent to a vinylene or ethynylene moiety.

Thus, the conjugated molecule comprises conjugated arylene and/or arylene-vinylene and/or arylene-ethynylene groups linked together to form a molecule that is generally conjugated along its backbone, which has at least one p/n junction, and which has an AC group at one end of the backbone. The conjugation of double bonds, including within an arylene group, and/or triple bonds along the backbone allows for the conductance of electrons.

The conductive path of the conjugated molecule may be optionally interrupted by the insertion of a spacer group, X, into the backbone, which in one embodiment is depicted by the general Formula II:

[Ar]_{q}—X—[Ar^{p}]—[Arⁿ]—[Ar]ᵣ-AC

Each Ar group is as defined above. In the depicted embodiment the spacer group occurs immediately before the p/n junction, but it will be appreciated that the spacer group may be placed between any two Ar groups in the chain, including between an Ar^{p} group and an Arⁿ group.

The spacer group, X, is non-conjugated, or partially non-conjugated. The spacer group serves to provide a break in the conjugation of the backbone, thereby allowing for further modulation of the electronic conductive properties of the conjugated molecule. For example, the spacer group could be a quantum well for charge transmission along the molecule, which will only allow electrons to be transmitted through the well from one side of the well to another at a certain potential. Thus, a skilled person will understand that the spacer group, if fully non-conjugated, or the non-conjugated portion of a partially non-conjugated spacer group, is not so large so as to prevent an electron from tunnelling through the spacer group, from conjugated backbone on one side of the spacer group to conjugated backbone on the other side of the spacer group.

The spacer group may be selected, for example, from methylene, ethylene, propenylene, ethylene dioxy, 1,4-cyclohexylene, 1,4-cyclohexylene dioxy, thio, dithio, thionyl, sulfonyl, imino, carbonyl, carbonyl dioxy, thiocarbonyl, phosphinidene and phosphonitryl, or a derivative of any of such groups, any of which methylene, ethylene, propenylene, ethylene dioxy, 1,4-cyclohexylene, 1,4-cyclohexylene dioxy may be substituted by C₁₋₈ alkyl, C₁₋₁₈ alkenyl, C₁₋₁₈ alkynyl, amido, carbonyl, sulfonyl, thioamide, C₅₋₃₀ aryl, C₅₋₃₀ arylamino, amino, nitro, cyano, isocyano and halide.

A skilled person will appreciate that more than one spacer group may be inserted along the conjugated backbone, provided that each spacer group is flanked by a sufficient number of Ar groups so as to allow for conductance of the electrical charge along the backbone, and across each spacer group. Where the conjugated molecule contains more than one spacer group, preferably from 1 to 20 Ar groups separate two adjacent spacer groups.

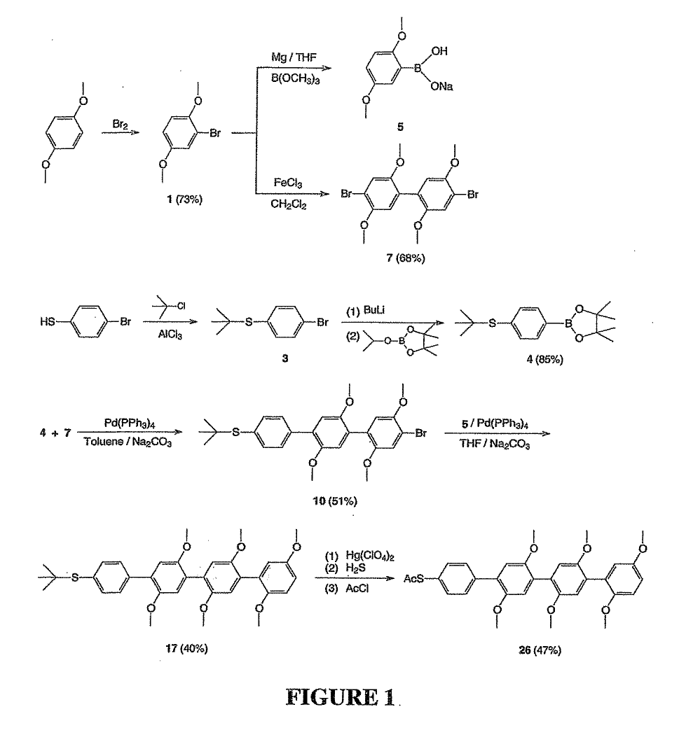

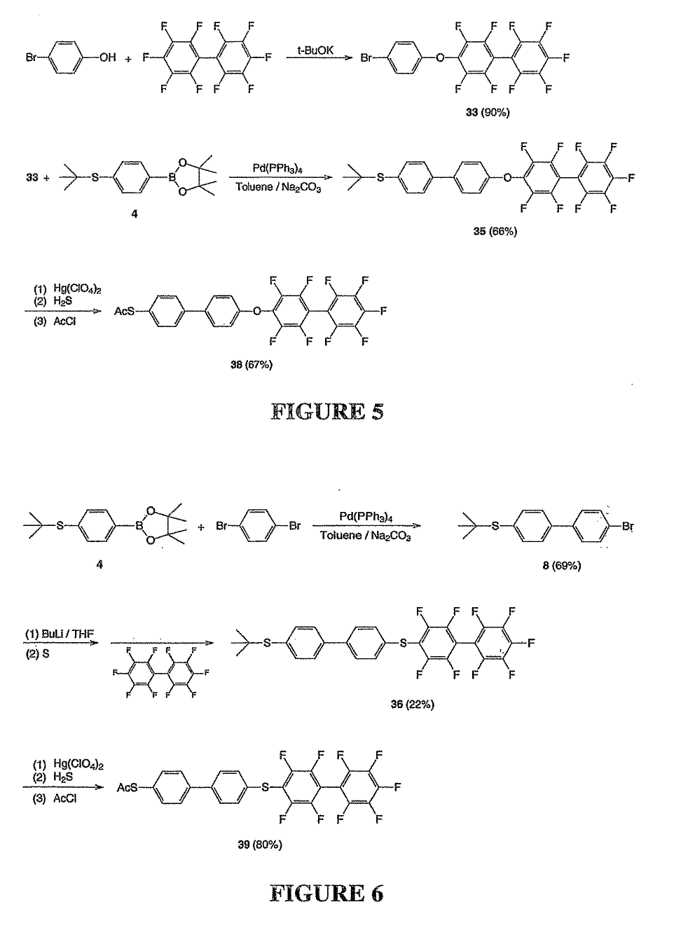

The conjugated molecules described herein are synthesized by coupling together individual Ar groups, spacer groups and an AC group, or the relevantly reactant molecules required to produce the desired carbon-carbon bond between groups, in a desired order so as to form a molecule having the particular sequence of Ar groups and spacer groups along the backbone, with an AC group at a particular end of the backbone, as required for a particular application. Thus, the individual Ar groups are added to the backbone in the desired order so as to form at least one p/n junction in the backbone, and so as to produce a conjugated molecule having the desired arrangement of p-segments, n-segments and spacer groups. FIGS. 1-6 set out schematic diagrams of exemplary synthesis mechanisms.

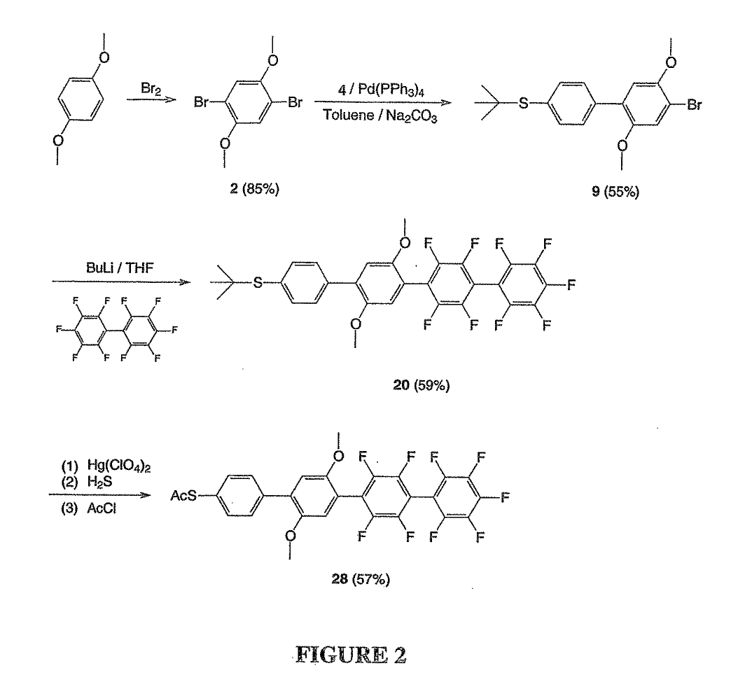

The conjugated molecules may be synthesized using standard methods known in the art. For example, the various Ar groups may be coupled together to form the conjugated backbone using standard carbon-carbon coupling reactions such as the Suzuki reaction. This approach can result in fairly high yields of product. Examples of such an approach are set out in the reaction schemes depicted in FIGS. 1 and 2. The Suzuki coupling reaction may typically yield more than 50% of mono-substituted product when there is more than one possible reactive site on the substrate. Other coupling reactions that may be used to connect Ar groups in the backbone, and which are known in the art, include the Grignard reaction, the Stille coupling reaction and the Heck reaction. A skilled person will understand that any nucleophilic substitution reaction that results in the formation of a carbon-carbon bond may be used to connect the various groups used to form a particular conjugated molecule of Formula I, or Formula II.

For example, 4′-tert-butylthiophenyl-4,4,5,5-tetramethyl-1,3,2-dioxaborolane, coupled with 2,5-dimethoxybromobenzene, catalyzed by Pd(PPh₃)₄, afford 4-tert-butylthio-2′,5′-dimethoxybiphenyl (as set out in Example 11 below). The same Suzuki reagent may be reacted with the larger substrate 4-bromo-2,2′,5,5′-tetramethoxybiphenyl to yield 4-(p-tert-butylthiophenyl)-2,2′,5,5′-tetramethoxybiphenyl (set out in Example 14).

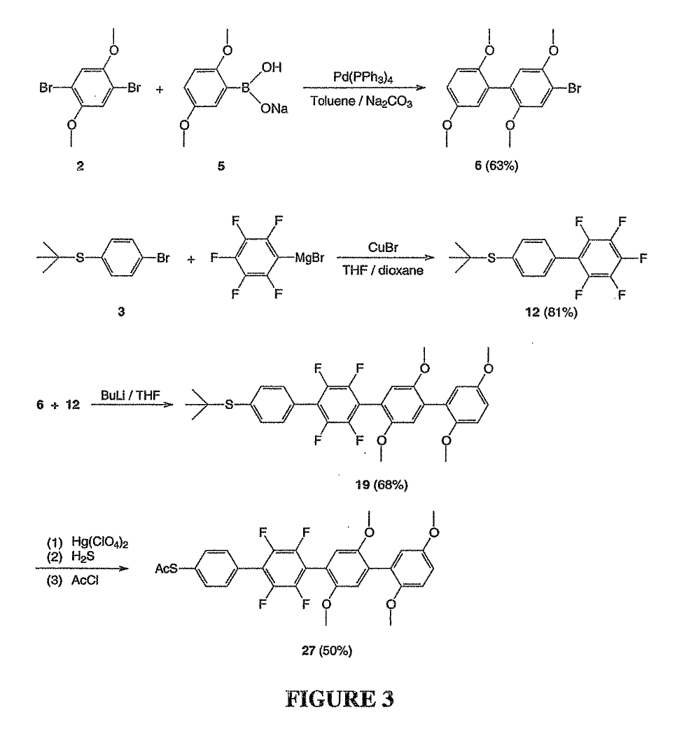

In another example, a perfluorophenyl group or perfluorobiphenyl group may be coupled with an aryl bromide compound using a perfluorophenyl Cu reagent as shown in FIG. 3. In such a coupling reaction, no catalyst is required and high yields may be achieved. 4-tert-butylthiophenyl-pentafluorobenzene (Example 12) may be prepared from pentafluorophenyl cuprous and 4-tert-butylthio-bromobenzene in THF and 1,4-dioxane. Similarly, 4-pentafluorophenyl-4′-tert-butylthio-2,5-dimethoxybiphenyl (see Example 16) may be prepared from pentafluorophenyl cuprous and 4′-tert-butylthio-4-bromo-2,5-dimethoxybiphenyl.

Perfluorophenyl or perfluorobiphenyl groups may also be incorporated into conjugated molecule through lithium nucleophilic substitution reaction, since such molecules possess one or more fluoro groups that may act as leaving groups on perfluorophenyl ring or biphenyl rings (see FIGS. 2 and 3). For example, p-tert-butylthiophenyl-nonafluorobiphenyl (Example 13) may be prepared by coupling of 4-tert-butylthiophenyl lithium in THF at −78° C. with decafluorobiphenyl. 1-tert-Butylthiophenyl-4-(2′,2″,5′,5″-tetramethoxybiphenyl)-tetrafluorobenzene (Example 19) may also be prepared following a similar procedure.

The synthesis of conjugated molecules containing arylene-ethynylene molecules is described in U.S. Pat. No. 6,756,605, issued to Reed et al., which is fully incorporated by reference herein.

The AC group, for example, a tert-butylthio group, may be introduced into the conjugated molecule using the standard carbon-carbon coupling reactions mentioned above, such as the Suzuki reaction. The preferred Suzuki reagent to incorporate a 4-tert-butylthiophenyl group onto the end of a conjugated molecule is 4-tert-butylthiophenyl-4,4,5,5-tetramethylborolane, which is prepared from 4-tert-butylthio-bromobenzene by reacting with butyl lithium and then 2-isopropyloxy-4,4,5,5-tetramethylborolane as shown in FIG. 1. This reagent may be used to react with most aryl bromides, and the reaction can be catalyzed by Pd(II) or Pd(0). Other Suzuki reagents that may be used include tert-butylthiophenyl boronic acid and methylthiophenyl boronic acid.

If an AC group containing a thio group, other than acetylthio, is used, such an AC group may be converted to acetylthio by mercurization to form a mercury sulfide group, followed by reaction with hydrogen sulfide and acetylation, as will be understood by a skilled person. Acetylthio is a preferred thio-containing AC group due to its reactivity, storage stability and ability to direct self-assembly of the conjugated molecule on a metal substrate.

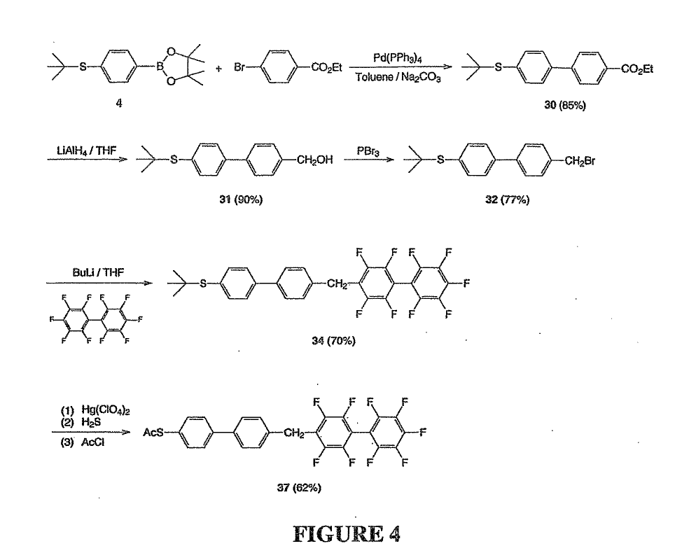

A skilled person will understand that the reaction used to introduce a spacer group will depend on the particular spacer group introduced. For example, the conjugated molecule having a spacer group may be prepared as depicted in FIGS. 4-6. For example, incorporation of a methylene spacer group in 4′-tert-butylthiobiphenyl-nonafluorobiphenyl-4-yl methane (Example 34) may be achieved by nucleophilic attack of 4-tert-butylthio-4′-bromomethyl-biphenyl on decafluorobiphenyl in the presence of butyl lithium. 4-tert-Butylthio-4′-bromomethyl-biphenyl (Example 32) was synthesized from the reduction product of ethyl 4-(p-tert-butylthiophenyl)-benzoate (Example 30) by bromination with phosphorous tribromide.

Similarly, an oxygen spacer group may be introduced from a phenol group by nucleophilic attack on perfluorobiphenyl in high yield.

Synthesis of a conjugated molecule having a sulfide spacer group is less efficient, as the yield of such compounds tends to be low, for example, 4′-tert-butylthiobiphenyl-4-yl-nonafluorobiphenyl-4-yl sulfide (see Example 36), which may be due in part to the weak nucleophilicity of the sulfide intermediate.

In use, the present conjugated molecules will only conduct current when under a forward bias. As with p/n junctions found in traditional silicon-based devices, the juxtaposition of an Ar^{p} group and Arⁿ group in the present molecules creates a interface across which electrons can flow in one direction but not in the other.

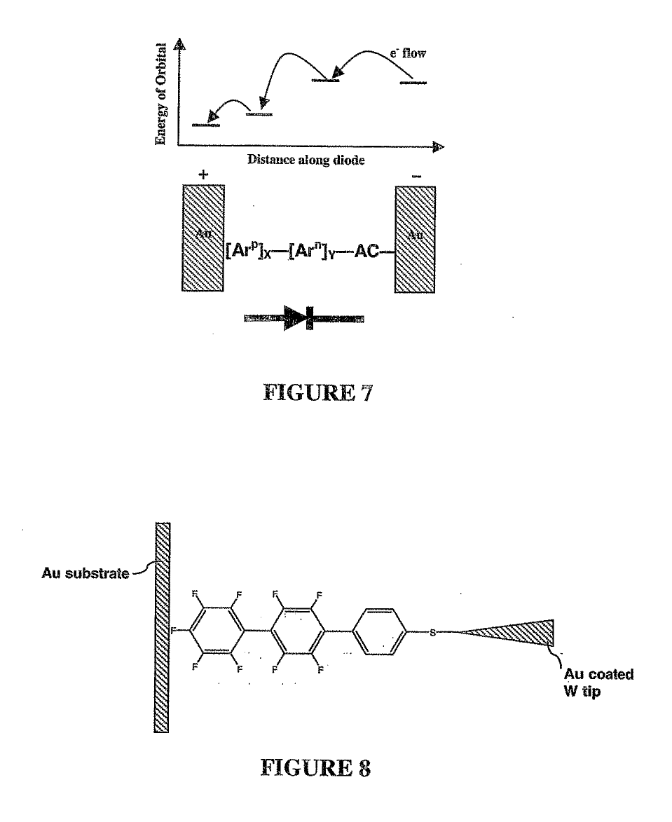

This direction of conductance is due to the difference in energy levels between the localized lowest unoccupied orbitals of the p-segment and the n-segment of the molecule, as is depicted in FIG. 7. Provided that the energy level of the orbitals associated with the Ar groups that are immediately adjacent to each electrical contact is different from the other, as will be the case where a p/n junction occurs, current will be able to flow in one direction across the molecule but not the other. When a positive bias is applied to a molecule having a p-segment next to the positive electrical contact and an n-segment next to the negative electrical contact, the Fermi energy level of the positive electrical contact will be close to the localized HOMO energy levels of the p-segment and holes could be injected into the HOMO of the p-segment from the negative electrical contact. Conversely, the Fermi energy level of the negative electrical contact will be raised and will be at similar energy levels to that of the localized LUMO adjacent to the negative contact. Thus, electrons can flow from the negative contact to the n-segment and will across the molecule to the positive contact. The electron or hole cannot overcome the energy barrier to flow in the opposite direction.

In the present conjugated molecule, the AC group facilitates attachment of the conjugated molecule to one electrode attachment within an electronic molecular device, typically a connection between the conjugated molecule and a metal or polymeric surface. The presence of the AC group at one of the molecule aids in the orientation of the molecule when assembling the molecule in an electronic molecular device. Since the conjugated molecule possesses a conductive direction, it is important to ensure proper orientation of the molecule when fabricating devices. As is discussed below, the AC group allows for the conjugated molecule to self-assemble on the surface of an electrode surface through a contact between the AC group and the electrode, ensuring that each molecule assembled on the electrode surface has the same orientation prior to forming a second electrical contact with the surface of a second electrode.

The inclusion of a spacer group in the present conjugated molecule allows for construction of negative differential resistance (NDR) devices, in which current will flow only within a given voltage range, and will not flow at voltages above and below this range.

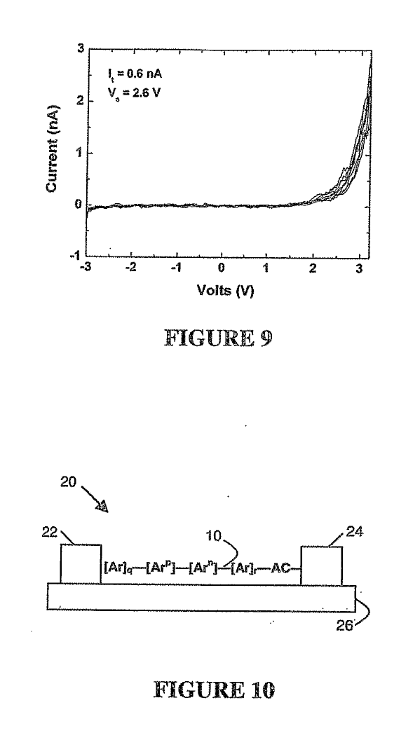

The electronic conducting property of the disclosed conjugated molecule can be measured using the conducting tip of a scanning tunnelling microscope, or using conducting atomic force microscopy, as will be understood by a skilled person. Briefly, the governing principle of scanning tunnelling microscopy (STM) is the quantum tunnelling of electrons through a thin potential barrier separating two electrodes where one electrode is a very sharp tip. By applying a voltage (Vt) between the tip and a metallic or semiconducting sample, a current can flow (It) between these electrodes when their distance is reduced to a few atomic diameters. The amplitude of the current strongly depends on the distance between the tip and the sample, and also on the potential difference Vt. By varying the potential difference between the tip and the sample, I(V) spectra as well as the differential conductance dI/dV(V) can be measured. The differential conductance reflects directly the local electronic density of states (LDOS). As shown in the schematic diagram FIG. 8, a conjugated molecule (which is depicted as 4-acetylthiophenyl-nonafluorobiphenyl) may be assembled on a gold substrate and coupled to a gold-coated tungsten tip of a scanning tunnelling microscope. The I-V characteristics measured for 4-acetylthiophenyl-nonafluorobiphenyl are illustrated by the graph of FIG. 9.

A conjugated molecule having at least one p/n junction, such as that described above, is useful for incorporation into various molecular electronic devices. Thus, as illustrated in FIG. 10, there is provided a molecular electronic device 20 having two electrical contacts 22 and 24. The first electrical contact 22 is connected to the second electrical contact 24 by conjugated molecule 10, so as to form a conductive path between the two electrical contacts.

The electrical contacts 22 and 24 may be composed of any conductive material, including any metal commonly used in electronic devices, such as gold, silver, copper, platinum or palladium. The electrical contacts 22 and 24 may alternatively be composed of indium tin oxide or a conductive polymeric material, for example poly(3,4-ethylenedioxythiophene) (PEDOT) or polyaniline. The first electrical contact may be composed of the same or different conductive material as the second electrical contact.

In order to provide a solid support for electrical contacts 22 and 24, substrate 26 may optionally be used. The electrical contacts may each be deposited on a thin layer on substrate 26, which may be silicon, mica or a plastic substrate such as polyethylene terephthalate or polycarbonate.

Asymmetric conjugated molecule 10 is coupled to electrical contact 24 through an interaction between the conductive material of electrical contact 24 and the AC group located at one end of the molecule. Electrical contact 22 is coupled to the first Ar group located in the backbone at the other end of conjugated molecule 10. The conjugated molecule 10 couples with electrical contacts 22 and 24 through formation of a bond with the material of electrical contact 24, such as a covalent or an ionic bond between the AC group and electrical contact 24, or through physical contact between the free Ar group end and electrical contact 22. The AC group on one end of conjugated molecule 10 may form a covalent bond with electrical contact 24 by first reacting to form a reactive group, such as a thiol group, which then reacts with the material of electrical contact 24.

The conductive path formed by conjugated molecule 10 may be a single molecule connected between electrical contacts 22 and 24, a plurality of molecules 10, or it may be formed from a monolayer formed of a plurality of conjugated molecule 10.

Depending on the design of the particular conjugated molecule 10 used in the device, device 20 may be suitable for various electronic applications.

For example, molecular electronic device 20 may be designed with diode characteristics by using conjugated molecule 10 having a p-type segment adjacent to an n-type segment within the same conductive path. The different type of segments will exhibit different electronic properties due to the character of the Ar groups and any substituents on the Ar groups included in the segment. Such a molecular electronic device will contain one p/n junction within the conductive path, analogous to a traditional microelectronic diode.

Molecular electronic device 20 may be designed to exhibit a negative differential resistance (NDR) effect when a suitable voltage is applied, thereby being suitable as a resonant tunnelling diode. For example, incorporation of a series of perfluorophenylene groups adjacent to a series of phenylene groups within conjugated molecule 10 results in molecular electronic device 20 that is an NDR device. Without being bound to a particular theory, this effect may be due to the large dihedron angle between the perfluorophenylene ring and the adjacent phenylene group. As well, the NDR effect may be ascribed to the redox process of the active molecules, as well as the conformational change that conjugated molecule 10 undergoes when placed under an electrical field. The redox behavior is directly relevant to the charge transport process in that a molecule that is more easily oxidized, or more easily reduced, can more easily conduct an electrical charge along the molecule. Thus, the redox behaviour of the conjugated molecule can be manipulated by incorporation of different segments with different electronic properties. Comparison of the redox behaviour of three conjugated molecules, namely 4-(p-tert-Butylthiophenyl)-2,2′,5,5′-tetramethoxybiphenyl (TSBOO), p-tert-Butylthiophenyl-nonafluorobiphenyl (TSBFF), tert-Butylthiophenyl-4-(2′,5′-dimethoxyphenyl)-tetrafluorobenzene (TSBFO) is illustrated in FIG. 11 (see Example 41 for details).

Alternatively, molecular electronic device 20 may be designed as a resonant tunnelling diode by the inclusion of conjugated molecule 10 having one or more non-conjugated spacer group X interspersed along the conjugated backbone. The non-conjugated spacer group inserted into the conjugated oligomer backbone creates a barrier for electron transport. Thus, in order for the device to conduct current at a certain applied voltage, it is necessary for an electron to tunnel, quantum mechanically, through the barrier.

Molecular electronic device 20 may be constructed as follows. Each electrical contact 22 and 24 may be deposited or patterned onto substrate 26. The conjugated molecule 10 is then assembled onto the first electrical contact 24 in a monolayer. This may be done, for example, by self-assembly of conjugated molecule 10. A skilled person will understand how to form self-assembled monolayers of the conjugated molecules. Briefly, substrate 26 having deposited electrical contact 24 is immersed in a solution of conjugated molecule 10 dissolved in a suitable organic solvent. The organic solvent is any solvent in which the particular molecule is soluble, and may be, for example, tetrahydrofuran, chloroform, dichloromethane, 1,2-dichloroethane, 1,1,2,2-tetrachloroethane, toluene, xylene, chlorobenzene, 1,2-dichlorobenzene, cyclohexanone or 2-methylfuran. Conjugated molecule 10 will arrange itself on the surface of electrical contact 24 with the AC group at the electrical contact/molecule interface, and the non-AC end free in solution. Addition of a base to the solution, for example sodium hydroxide or ammonium hydroxide, may help to promote the self-assembly process. Alternatively, Langmuir-Blodgett techniques, which are known in the art, may be used to form a monolayer of conjugated molecule 10 on electrical contact 24. Electrical contact 22 is then deposited over top of the monolayer of conjugated molecule 10.

Molecular electronic device 20 may be assembled into larger electronic devices for various applications, including memory devices and sensors. For example, the resonant tunnelling diodes described herein are useful as molecular switches and can also be integrated into logic gates. NDR devices have numerous applications, including high frequency oscillators, multipliers, logic gates, analog-to-digital converters.

For example, the molecular electronic device may be used to form a crossbar, and may be assembled into crossbar devices. Crossbars and crossbar devices are known in the art, and are described in U.S. Pat. Nos. 6,459,095 and 6,128,214, both of which are herein fully incorporated by reference. A crossbar is an electronic connection that connects two crossed conductors 32 and 34, for example, wires, which intersect at a non-zero angle, for example, a right angle. In this case, the electronic connection is molecular electronic device 20 in which the electrical contacts are at non-zero angles to each other. Preferably, the electrical contacts are at right angles to each other. Molecular electronic device 20 used in the crossbar device may be a molecular switch, a diode, a transistor or the like, and serves to connect the crossed conductors at the point at which they intersect.

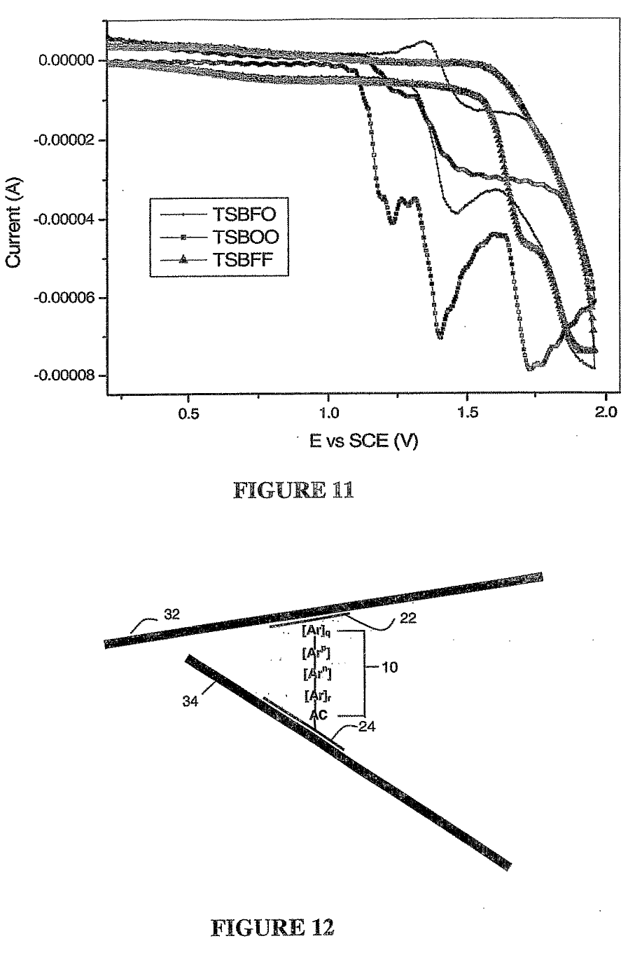

A crossbar containing molecular electronic device 20 is depicted in FIG. 12. A series of crossbars may be arranged in an array to form a crossbar device, which comprises a series of molecular electronic devices connecting two sets of crossed parallel conductors. Each set of parallel conductors generally forms a plane, and the two sets are separated from each other, and are arranged so that the conductors in each set cross the conductors in the other. At each crossover point between the two sets of conductors, a conductor from each set is connected to a conductor from the other set by an electronic connection, in this case, by molecular electronic device 20. Such crossbar devices are useful as memory devices and sensors.

All documents referred to herein are fully incorporated by reference.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. All technical and scientific terms used herein have the same meaning as commonly understood by one of ordinary skill in the art of this invention, unless defined otherwise.

The following examples illustrate reactions for coupling various Ar groups, spacer groups and AC groups, which reactions and variations thereof may be used to synthesize particular embodiments of the conjugated molecules.

### EXAMPLES

### Example 1

### Preparation of 2,5-dimethoxybromobenzene (1)

In a one liter round-bottom flask, a solution of bromine (16.0 g, 100 mmol) in acetic acid (50 ml) was added dropwise to a solution of 1,4-dimethoxybenzene (13.8 g, 100 mmol) in chloroform and methanol (400 ml) at 0° C. After stirring for 3 hours, 500 ml of saturated Na₂CO₃ solution was added. The organic layer was washed with water, brine and dried over sodium sulfate. After the solvent was removed on a rotary evaporator, the residue was distilled under reduced pressure to yield 15.8 g of 2,5-dimethoxybromobenzene (yield 73%) ¹HNMR (CDCl₃) δ7.148 (s, 1H), 6.859 (s, 2H), 3.869 (s, 3H), 3.785 (s, 3H).

### Example 2

### Preparation of 2,5-dibromo-1,4-dimethoxybenzene (2)

In a one liter round-bottom flask, a solution of bromine (35.2 g, 220 mmol) in chloroform (50 ml) was added dropwise to a solution of 1,4-dimethoxybenzene (13.8 g, 100 mmol) in chloroform (400 ml) under 0° C. After stirring for 3 hours, 100 ml of saturated Na₂CO₃ solution was added. The organic layer was washed with water, brine, and dried over sodium sulfate. The solvent was removed on a rotary evaporator and the residue was performed recrystallization from ethanol to afford pure 2,5-dibromo-1,4-dimethoxybenzene (25.8 g, 85%). ¹HNMR (CDCl₃) δ7.128 (s, 2H), 3.873 (s, 6 H).

### Example 3

### Preparation of 4-tert-butylthio-bromobenzene (3)

In a 50 ml round-bottom flask, a mixture of 20 ml of tert-butyl chloride and 9.5 g of 4-bromothiophenol was stirred until all the solids dissolved, then a catalytic amount of AlCl₃ was added. After stirring for 2 hours, the mixture was poured into 100 ml of ice water and extracted with hexane twice. The solvent was removed and the crude product was purified with flash column (used hexane as the eluent) to offer 10.5 g of 4-tert-butylthio-bromobenzene. The obtained product was used in synthesis reactions without further purification.

### Example 4

### Preparation of 4-tert-butylthiophenyl-4,4,5,5-tetramethyl-1,3,2-dioxaborolane (4)

In a 150 ml round-bottom flask, a solution of 4.9 g (20 mmol) of 4-tert-butylthio-bromobenzene in 60 ml of THF was cooled to −78° C.; 24 ml of butyl lithium (1.25 M) in 20 ml of THF was then slowly added dropwise to a round-bottom flask. After stirring for 1 hour, 5.58 g (30 mmol) of 2-isopropoxy-4,4,5,5-tetramethyl-1,3,2-dioxaborolane was added in one portion. The temperature was raised to room temperature and stirred overnight. The reaction was terminated and was washed with saturated sodium bicarbonate solution and brine. The organic phase was dried with sodium sulfate and the solvent was removed on a rotary evaporator. The residue was purified by column with hexane and CH₂Cl₂ (4:1) to offer 4.9 g (yield 85%) of 4-tert-butylthiophenyl-4,4,5,5-tetramethyl-1,3,2-dioxaborolane. ¹HNMR (CDCl₃) δ7.880 (d, 2H, J=8.0 Hz), 7.600 (d, 2H, J=8.0 Hz), 1.468 (s, 12H), 1.407 (s, 9H).

### Example 5

### Preparation of sodium 2,5-dimethoxyphenylboronate (5)

In a 250 ml 3-neck round-bottom flask, 1.44 g of Mg, 10 ml of THF and 0.8 g of 2,5-dimethoxybromobenzene was stirred rapidly. After the initiation of the reaction, a solution of 10 g of 2,5-dimethoxybromobenzene in 90 ml of THF was added dropwise to a round-bottom flask under nitrogen. The reaction was refluxed for another two hours, then cooled down to room temperature and the solution was transferred into another round-bottom flask containing a solution of 10 g of trimethyl borate in 300 ml of THF which had been cooled to −78° C. The reaction mixture was gradually warmed up to room temperature and stirred overnight. 200 ml of 2 M HCl was added to quench the reaction and stirred for 1 hour. After THF was removed on a rotary evaporator, the residue was extracted with 200 ml ether. 5 M NaOH was added dropwise to the organic phase until no more solid came out. The white solid was filtered and dried under vacuum to yield 9.0 g sodium 2,5-dimethoxyphenylboronate. The solid was used for synthesis without further purification.

### Example 6

### Preparation of 4-bromo-2,2′,5,5′-tetramethoxybiphenyl (6)

In an argon flushed two-neck round-bottom flask, a mixture of 1.02 g (5.0 mmol) of sodium 2,5-dimethoxyphenylboronate, 3.0 g (10 mmol) of 2,5-dibromo-1,4-dimethoxybenzene, 60 mg (1 mol %) of tetrakis(triphenylphosphine)palladium, 20 ml of 2 M sodium carbonate and 50 ml of THF was added and heated at reflux for two hours. After cooling, the reaction mixture was extracted with ethyl acetate and the organic phase was washed with brine and dried over magnesium sulfate. After the solvent was removed on a rotary evaporator, the residue was purified by column chromatography eluted with hexane/CH₂Cl₂ (4:1) to give 1.0 g 4-bromo-2,2′,5,5′-tetramethoxybiphenyl (yield 63%). ¹HNMR (CDCl₃) δ7.187 (s, 1H), 6.836-6.951 (m, 4H), 3.869 (s, 3H), 3.814 (s, 3H), 3.757 (s, 6H). ¹³CNMR (CDCl₃) δ153.788, 151.830, 151.596, 150.278, 128.277, 127.995, 117.615, 117.293, 115.897, 113.924, 112.933, 110.019, 57.291, 57.042, 56.897, 56.171.

### Example 7

### Preparation of 4,4′-dibromo-2,2′,5,5′-tetramethoxybiphenyl (7)

In a 100 ml round-bottom flask, 3.0 g of FeCl₃ in 20 ml of CH₂Cl₂ was added to a solution of 5.0 g of 2,5-dimethoxybromobenzene in 50 ml of CH₂Cl₂. The reaction mixture was stirred overnight, and then it was washed with water and brine and dried with magnesium sulfate. The solvent was removed on a rotary evaporator and the residue was purified by column chromatography eluted by hexane/CH₂Cl₂ (3:1) to obtain 3.4 g of 4,4′-dibromo-2,2′,5,5′-tetramethoxybiphenyl (yield 68%) ¹HNMR (CDCl₃) δ7.194 (s, 2H), 6.841 (s, 2H), 3.877 (s 3H), 3.757 (s, 3H).

### Example 8

### Preparation of 4-tert-butylthio-4′-bromobiphenyl (8)

In an argon flushed two neck round-bottom flask, a mixture of 1.46 g (5.0 mmol) of 4-tert-butylthiophenyl-4,4,5,5-tetramethyl-1,3,2-dioxaborolane, 2.36 g (10 mmol) of 1,4-dibromobenzene, 90 mg (1.5 mol %) of tetrakis(triphenylphosphine)palladium, 20 ml of 2 M sodium carbonate and 50 ml of toluene was stirred under 90° C. overnight. After cooling down, it was extracted with ethyl acetate twice and the organic phase was washed with brine and dried with magnesium sulfate. The solvent was removed on a rotary evaporator, the residue was purified by column eluted with hexane/CH₂Cl₂ (5:1) to offer 1.10 g of 4-tert-butylthio-4′-bromobiphenyl (yield 69%). ¹HNMR (CDCl₃) δ7.631-7.597 (m, 4H), 7.551 (d, 2H), 7.161 (d, 2H), 1.347 (s, 9H).

### Example 9

### Preparation of 4′-tert-butylthio-4-bromo-2,5-dimethoxybiphenyl (9)

In an argon flushed two neck round-bottom flask, a mixture of 1.46 g (5.0 mmol) of 4-tert-butylthiophenyl-4,4,5,5-tetramethyl-1,3,2-dioxaborolane, 2.0 g (6.7 mmol) of 2,5-dibromo-1,4-dimethoxybenzene, 90 mg (1.5 mol %) tetrakis(triphenylphosphine)palladium, 20 ml of 2 M sodium carbonate and 50 ml of toluene was stirred under 85° C. overnight. After cooling down, it was extracted with ethyl acetate and washed with brine and dried with magnesium sulfate. The solvent was removed on a rotary evaporator, the residue was purified by column eluted first by hexane followed by the mixture of hexane and CH₂Cl₂ (3:1) to offer 1.04 g of 4′-tert-butylthio-4-bromo-2,5-dimethoxybiphenyl (yield 55%). ¹HNMR (CDCl₃) δ7.604 (d, 2H, J=8.0 Hz), 7.505 (d, 2H, J=8.0 Hz), 7.203 (s, 1H), 6.919 (s, 1H), 3.913 (s, 3 H), 3.793 (s, 3H), 1.359 (s, 9H). ¹³CNMR (CDCl₃) δ151.233, 150.744, 138.523, 137.534, 132.179, 130.241, 129.753, 117.506, 115.180, 111.291, 57.389, 56.870, 46.483, 31.433.

### Example 10

### Preparation of 4′-(p-tert-butylthiophenyl)-4-bromo-2,2′,5,5′-tetramethoxybiphenyl (10)

In an argon flushed two neck round-bottom flask, a mixture of 1.46 g (5.0 mmol) of 4-tert-butylthiophenyl-4,4,5,5-tetramethyl-1,3,2-dioxaborolane, 3.0 g (6.9 mmol) of 4,4′-dibromo-2,2′,5,5′-tetramethoxybiphenyl, 90 mg (1.5 mol %) of tetrakis(triphenylphosphine)palladium, 20 ml of 2 M sodium carbonate and 50 ml of toluene was stirred under 85° C. overnight. After cooling down, it was extracted with ethyl acetate and washed with brine and dried with magnesium sulfate. The solvent was removed on a rotary evaporator, the residue was purified by column eluted with hexane followed by the mixture of hexane and CH₂Cl₂=10:1 and finally with hexane:CH₂Cl₂=3:1 to offer 1.31 g 4′-(p-tert-butylthiophenyl)-4-bromo-2,2′,5,5′-tetramethoxybiphenyl (yield 51%). ¹HNMR (CDCl₃) δ7.624 (d, 2H, J=8.0 Hz), 7.588 (d, 2H, J=8.0 Hz), 7.229 (s, 1H), 6.996 (s, 1H), 6.944 (s, 1H), 6.933 (s, 1H), 3.902 (s, 3H), 3.807-3.799 (d, 9 H), 1.375 (s, 9H). ¹³CNMR (CDCl₃) δ151.847, 151.472, 150.688, 150.351, 139.199, 137.490, 131.794, 130.401, 129.915, 127.696, 127.207, 117.363, 116.005, 115.607, 114.950, 114.214, 57.367, 57.097, 57.007, 56.795, 46.444, 31.453.

### Example 11

### Preparation of 4-tert-butylthio-2′,5′-dimethoxybiphenyl (11)

In an argon flushed two neck round-bottom flask, a mixture of 1.46 g (5.0 mmol) of 4′-tert-butylthiophenyl-4,4,5,5-tetramethyl-1,3,2-dioxaborolane, 1.2 g (5.5 mmol) of 2,5-dimethoxybromobenzene, 90 mg (1.5 mol %) of tetrakis(triphenylphosphine)palladium, 20 ml of 2 M sodium carbonate and 50 ml of toluene was heated at reflux overnight. After cooling down, the reaction mixture was extracted with ethyl acetate and the organic phase was washed with brine and dried over magnesium sulfate. The solvent was removed on a rotary evaporator and the residue was purified by column eluted with hexane/CH₂Cl₂ (5:1) to offer 1.16 g of 4-tert-butylthio-2′,5′-dimethoxybiphenyl (yield 77%). ¹HNMR (CDCl₃) δ7.594 (d, 2H, J=8.0 Hz), 7.532 (d, 2H, J=8.0 Hz), 6.958-6.898 (m, 3H), 3.834 (s, 3H), 3.784 (d, 3H), 1.357 (s, 9H). ¹³CNMR (CDCl₃) δ154.252, 151.190, 139.261, 137.455, 131.754, 131.224, 129.938, 117.053, 113.894, 113.220, 56.715, 56.208, 46.573, 31.471.

### Example 12

### Preparation of 4-tert-butylthiophenyl-pentafluorobenzene (12)

10 ml of Pentafluorophenylmagnesium bromide (0.5 M in ether, 5 mmol) was added into the mixture of 2.88 g of CuBr and 10 ml of THF in a 100 ml round-bottom flask under room temperature. After stirring for one hour, 5 ml of 1,4-dioxane and 0.74 g (3 mmol) of 4-tert-butylthio-bromobenzene in 5 ml of toluene were added. The mixture was stirred at 90° C. overnight. The reaction was quenched with 2 M HCl and the salt was filtered through Celite. The solvent was removed on a rotary evaporator and the residue was purified by column eluted with hexane to give 0.81 g of 4-tert-butylthiophenyl-pentafluorobenzene (yield 81%). ¹HNMR (CDCl₃) δ7.685 (d, 2H, J=8.0 Hz), 7.420 (d, 2H, J=8.0 Hz), 1.365 (s, 9H). ¹³CNMR (CDCl₃) δ151.750, 150.750, 138.660, 137.527, 132.711, 132.338, 129.873, 115.638, 115.114, 114.657, 56.315, 46.516, 31.446.

### Example 13

### Preparation of p-tert-Butylthiophenyl-nonafluorobiphenyl (13)

In an argon flushed 25 ml round-bottom flask, a solution of 0.49 g of 4-tert-butylthio-bromobenzene (2 mmol) in 10 ml of THF was stirred in a dry ice-acetone bath until the temperature was dropped to −78° C., then 1.6 ml of butyl lithium (1.25 M in hexane) was slowly injected to keep the temperature below −30° C., the reaction was sustained at −50° C. for 1 hour then the temperature was raised to 0° C. for 10 mins, then cooled down to −78° C. again and 1.0 g of decafluorobiphenyl (3 mmol) in 5 ml of THF was injected in one portion. The temperature was slowly raised to room temperature and kept stirring overnight. 10 ml of saturated sodium bicarbonate solution was added to quench the reaction and the mixture was extracted with ethyl acetate (5 ml×3). The organic phase was washed with brine and dried over magnesium sulfate. The solvent was removed on a rotary evaporator and the residue was purified by column eluted with hexane to offer 0.61 g of p-tert-butylthiophenyl-nonafluorobiphenyl (yield 63%). ¹HNMR (CDCl₃) δ7.525 (d, 2H, J=8.0 Hz), 7.521 (d, 2H, J=8.0 Hz), 1.382 (s, 9H). ¹³CNMR (CDCl₃) δ137.799, 135.456, 130.452, 127.406, 46.972, 31.475.

### Example 14

### Preparation of 4-(p-tert-butylthiophenyl)-2,2′,5,5′-tetramethoxybiphenyl (14)

In an argon flushed two neck round-bottom flask, a mixture of 1.46 g (5.0 mmol) of 4-tert-butylthiophenyl-4,4,5,5-tetramethyl-1,3,2-dioxaborolane, 2.0 g (5.7 mmol) of 4-bromo-2,2′,5,5′-tetramethoxybiphenyl, 90 mg (1.5 mol %) of tetrakis(triphenylphosphine)palladium, 20 ml of 2 M sodium carbonate and 50 ml of toluene was heated at reflux overnight. After cooling down, it was extracted with ethyl acetate and the organic phase washed with brine and dried over magnesium sulfate. The solvent was removed on a rotary evaporator, the residue was purified by column eluted with hexane/CH₂Cl₂ (5:1) to offer 1.51 g of 4-(p-tert-butylthiophenyl)-2,2′,5,5′-tetramethoxybiphenyl (yield 69%). ¹HNMR (CDCl₃) δ7.598-7.592 (m, 4H), 6.989 (s, 1H), 6.960-6.930 (m, 3H), 3.833 (s, 3H), 3.805 (s, 6H), 3.790 (s, 3H), 1.371 (s, 9H). ¹³CNMR (CDCl₃) δ153.842, 151.770, 151.621, 150.670, 139.401, 137.489, 131.648, 129.982, 128.829, 128.104, 117.786, 115.754, 114.916, 113.849, 112.954, 57.032, 56.971, 56.730, 56.192, 46.426, 31.840.

### Example 15

### Preparation of tert-butylthiophenyl-4-(2′,5′-dimethoxyphenyl)-tetrafluorobenzene (15)

In a 50 ml round-bottom flask, a solution of 0.434 g of 2,5-dimethoxybromobenzene (2 mmol) in 20 ml of THF cooled to −78° C. under N₂ atmosphere was injected 0.75 ml of butyl lithium (1.6 M in hexane, 1.2 mmol). Kept the temperature at −78° C. with stirring for 1 hour. Then 0.332 g of 4-tert-butylthiophenyl-pentafluorobenzene (1 mmol) in 10 ml of THF was injected in one portion. Raised the temperature to room temperature and stirred overnight. 5 ml of 2 M HCl was added to quench the reaction and extracted with 20 ml×3 of ethyl acetate and dried with magnesium sulfate. The solvent was then removed on a rotary evaporator. The residue was purified by column eluted with hexane:CH₂Cl₂ (3:1) to give 0.260 g of tert-butylthiophenyl-4-(2′,5′-dimethoxyphenyl)-tetrafluorobenzene (yield 50%). ¹HNMR (CDCl₃) δ7.699 (d, 2H, J=8.0 Hz), 7.520 (d, 2H, J=8.0 Hz), 7.022 (s, 2H), 6.901 (s, 1H), 3.837 (s, 6H), 1.378 (s, 9H). ¹³CNMR (CDCl₃) δ153.804, 151.835, 137.727, 134.511, 130.597, 128.456, 117.629, 116.335, 112.875, 56.776, 56.273, 46.840, 31.480.

### Example 16

### Preparation of 4-pentafluorophenyl-4′-tert-butylthio-2,5-dimethoxybiphenyl (16)

20 ml of Pentafluorophenylmagnesium bromide (0.5 M in ether, 10 mmol) was added into a mixture of 2.88 g CuBr and 10 ml of THF in a 100 ml round-bottom flask under room temperature, after stirring for one hour, 5 ml of 1,4-dioxane and 1.91 g (5 mmol) of 4′-tert-butylthio-4-bromo-2,5-dimethoxybiphenyl in 10 ml of toluene were added. The mixture was stirred at 90° C. overnight. The reaction was quenched with 2 M HCl and the salts were filtered out through Celite. The solvent was removed on a rotary evaporator. The residue was purified by column eluted with hexane/CH₂Cl₂ (4:1) to give 1.45 g 4-pentafluorophenyl-4′-tert-butylthio-2,5-dimethoxybiphenyl (yield 62%). ¹HNMR (CDCl₃) δ7.634 (d, 2H, J=8.0 Hz), 7.575 (d, 2H, J=8.0 Hz), 7.022 (s, 1H), 6.876 (s, 1H), 3.831 (s, 3H), 3.793 (s, 3H), 1.372 (s, 9H). ¹³CNMR (CDCl₃) δ154.252, 151.190, 139.261, 137.455, 131.754, 131.224, 129.938, 117.053, 113.894, 113.220, 56.715, 56.208, 46.573, 31.471.

### Example 17

### Preparation of 4-(2,5-dimethoxyphenyl)-4′-(p-tert-butylthio)phenyl-2,2′,5,5′-tetramethoxybiphenyl (17)

In an argon flushed two neck round-bottom flask, a mixture of 1.0 g of 4′-(p-tert-butylthiophenyl)-4-bromo-2,2′,5,5′-tetramethoxybiphenyl (2 mmol), 1.0 g of sodium 2,5-dimethoxyphenylboronate, 90 mg (2.0 mol %) of tetrakis(triphenylphosphine)palladium, 20 ml of saturated sodium bicarbonate solution and 50 ml of THF were heated at reflux overnight. After cooling down, the reaction mixture was extracted with ethyl acetate and the organic phase was washed with brine and dried over magnesium sulfate. The solvent was removed on a rotary evaporator and the residue was purified by column eluted with hexane/CH₂Cl₂ (2:1) to offer 0.46 g of 4-(2,5-dimethoxyphenyl)-4′-(p-tert-butylthio)phenyl-2,2′,5,5′-tetramethoxybiphenyl (yield 40%). ¹HNMR (CDCl₃) δ7.609 (s, 4H), 7.052-6.931 (m, 7H), 3.854-3.791 (m, 18H), 1.381 (s, 9H). ¹³CNMR (CDCl₃) δ140.755, 139.084, 138.966, 138.590, 138.562, 138.146, 128.780, 127.192, 122.322, 120.945, 120.189, 119.417, 119.198, 118.804, 110.823, 109.317, 109.017, 108.971, 108.437, 107.534, 106.764, 60.326, 60.260, 60.249, 60.290, 59.608, 51.498, 39.049.

### Example 18

### Preparation of 4-pentafluorophenyl-4′-(p-tert-butylthio)phenyl-2,2′,5′5′-tetramethoxybiphenyl (18)

10 ml of Pentafluorophenylmagnesium bromide (0.5 M in ether, 5 mmol) was added into a mixture of 2.88 g of CuBr and 10 ml of THF in a 100 ml round-bottom flask under room temperature. After stirring for one hour, 5 ml of 1,4-dioxane and 1.56 g (3 mmol) of 4′-(p-tert-butylthiophenyl)-4-bromo-2,2′,5,5′-tetramethoxybiphenyl in 10 ml of toluene were added. The mixture was stirred at 90° C. overnight. The reaction was quenched with 2 M HCl and the salts were filtered out through Celite. The solvent was removed on a rotary evaporator. The residue was purified by column eluted with hexane:CH₂Cl₂ (4:1) to give 0.76 g of 4-pentafluorophenyl-4′-(p-tert-butylthio)phenyl-2,2′,5′5′-tetramethoxybiphenyl (yield 42%). ¹HNMR (CDCl₃) δ7.609 (d, 2H, J=8.0 Hz), 7.601 (d, 2H, J=8.0 Hz), 7.043 (s, 1H), 7.022 (s, 1H), 7.009 (s, 1H), 6.894 (s, 1H), 3.845 (s, 3H), 3.817-3.810 (d, 9H), 1.376 (s, 9H). ¹³CNMR (CDCl₃) δ151.509, 151.368, 151.326, 150.690, 139.210, 137.500, 131.782, 130.429, 130.268, 129.935, 127.361, 115.698, 115.439, 115.035, 114.937, 57.051, 57.008, 56.797, 56.736, 46.547, 31.446.

### Example 19

### Preparation of 1-tert-butylthiophenyl-4-(2′,2″,5′,5″-tetramethoxybiphenyl)-tetrafluorobenzene (19)

In a 50 ml round-bottom flask, to a solution of 0.71 g of 4-bromo-2,2′,5,5′-tetramethoxybiphenyl (2 mmol) in 20 ml of THF cooled to −78° C. under N₂ atmosphere was injected 0.75 ml of butyl lithium (1.6 M in hexane, 1.2 mmol). Kept the temperature at −78° C. with stirring for 1 hour. Then 0.332 g of 4-tert-butylthiophenyl-pentafluorobenzene (1.0 mmol) in 10 ml of THF was injected in one portion. Raised the temperature to room temperature and stirred overnight. 5 ml of 2 M HCl was added to quench the reaction and extracted with 20 ml×3 of ethyl acetate and dried with magnesium sulfate. The solvent was then removed on a rotary evaporator. The residue was purified by column eluted with hexane/CH₂Cl₂ (2:1) to give 0.40 g 1-tert-butylthiophenyl-4-(2′,2″,5′,5″-tetramethoxybiphenyl)-tetrafluorobenzene (yield 68%). ¹HNMR (CDCl₃) δ7.713 (d, 2H, J=8.0 Hz), 7.538 (d, 2H, J=8.0 Hz), 7.018-6.949 (m, 5H), 3.842 (s, 6H), 3.816 (s, 3H), 3.791 (s, 3H), 1.388 (s, 9 H). ¹³CNMR (CDCl₃) δ153.823, 151.679, 151.393, 151.332, 137.739, 134.521, 130.613, 130.437, 128.513, 117.24, 115.967, 115.507, 115.388, 114.156, 112.986, 57.014, 56.927, 56.809, 56.202, 46.846, 31.489.

### Example 20

### Preparation of 4-(2′,5′-dimethoxy-4″-tert-butylthiophenyl)phenyl-nonafluorobiphenyl (20)

To a solution of 0.76 g of 4′-(tert-butylthio)-4-bromo-2,5-dimethoxybiphenyl (2 mmol) in 30 ml of THF at −78° C. under N₂ atmosphere was added 2 ml of butyl lithium (1.25 M in hexane, 2.5 mmol). The reaction mixture was kept at −50° C. with stirring for 1 hour then transferred to a solution of 1.0 g of decafluorobiphenyl (3 mmol) in 20 ml of THF under −50° C. The temperature was raised to room temperature and stirred overnight. 20 ml of saturated sodium bicarbonate solution was added to quench the reaction and the mixture was extracted with 20 ml of ethyl acetate twice and dried over magnesium sulfate. The solvent was then removed on a rotary evaporator. The residue was purified by column eluted with hexane to offer 0.73 g (yield 59%) of 4-(2′,5′-dimethoxy-4″-tert-butylthiophenyl)phenyl-nonafluorobiphenyl. ¹HNMR δ7.649 (CDCl₃) (d, 2H, J=8.0 Hz), 7.601 (d, 2H, J=8.0 Hz), 7.068 (s, 1H), 6.966 (s, 1H), 3.886 (s, 3H), 3.824 (s, 3H), 1.360 (s, 9H). ¹³CNMR (CDCl₃) δ151.69, 150.78, 138.63, 137.54, 132.98, 132.40, 129.89, 115.50, 115.37, 114.73, 56.882, 46.531, 31.447.

### Example 21

### Preparation of 4′-tert-butylthiobiphenyl-4-yl-nonafluorobiphenyl (21)

In an argon flushed 25 ml round-bottom flask, a solution of 0.64 g of 4-tert-butylthio-4′-bromo-biphenyl (2 mmol) in 10 ml of THF was stirred in a dry ice-acetone bath until the temperature was dropped to −78° C., then 1.6 ml of butyl lithium (1.25 M in hexane) was slowly injected while the temperature was kept below −30° C. The mixture was stirred for 1 hour at −50° C., then raised to 0° C. and stirred for another 10 mins, then cooled the system back to −78° C. again and 1.0 g of decafluorobiphenyl (3 mmol) in 5 ml of THF was injected in one portion. Raised the temperature slowly to room temperature and stirred overnight. 10 ml of saturated sodium bicarbonate solution was added to quench the reaction and the mixture was extracted with ethyl acetate (5 ml×3). The organic phase was washed with brine and dried over magnesium sulfate. The solvent was removed on a rotary evaporator, the residue was purified by column eluted with hexane to offer 0.50 g of 4′-tert-butylthiobiphenyl-4-yl-nonafluorobiphenyl (yield 45%). ¹HNMR (CDCl₃) δ 7.802 (d, 2H), 7.654-7.634 (m, 6H), 1.365 (s, 9H). ¹³CNMR (CDCl₃) δ142.066, 140.720, 138.316, 133.056, 131.004, 127.791, 127.548, 46.628, 41.427

### Example 22

### Preparation of p-acetylthiophenyl-nonafluorobiphenyl (22)

In a 50 ml two neck round-bottom flask, 0.24 g (0.5 mmol) p-tert-butylthiophenyl-nonafluorobiphenyl was dissolved in 20 ml of chloroform and then a solution of 0.4 g (1 mmol) mercury (II) perchlorate hydrate in 10 ml of methanol was added dropwise. Yellow color solids came out when enough mercury salts were added and the system was stirred overnight under room temperature. Then H₂S was bubbled into the reaction system with argon slowly until all the yellow solids turned into dark. The solids were filtered through Celite. The filtrate was washed with brine and dried with sodium sulfate. The solvent was removed on a rotary evaporator, and the residue was dissolved in 20 ml of chloroform and 0.5 ml of triethyl amine and 1 ml of acetic chloride were added and stirred for 10 mins. Methanol (3 ml) was added to quench the reaction. The reaction mixture was washed with water, 2 M sodium carbonate, and brine and the organic phase was dried with sodium sulfate. The solvent was removed on a rotary evaporator, the residue was purified by column eluted with hexane/CH₂Cl₂ (4:1) to offer 150 mg of p-acetylthiophenyl-nonafluorobiphenyl (yield 64%). ¹HNMR (CDCl₃) δ7.605 (s, 4 H), 2.504 (s, 3H). ¹³CNMR (CDCl₃) δ193.543, 134.853, 130.387, 30.800.

### Example 23

### Preparation of 4-(p-acetylthiophenyl)-2,2′,5,5′-tetramethoxy-biphenyl (23)

In a 50 ml two neck round-bottom flask, 0.22 g (0.5 mmol) 4-(p-tert-butylthiophenyl)-2,2′,5,5′-tetramethoxy-biphenyl was dissolved in 20 ml of chloroform and then a solution of 0.4 g (1 mmol) mercury(II) perchlorate hydrate in 10 ml of methanol was added drop by drop and yellow solids came out when enough mercury salts were added. The system was stirred overnight under room temperature. Then H₂S was bubbled in with argon slowly until all the yellow solids turned into dark. The solids were filtered through Celite. The filtrate was washed with brine and dried with sodium sulfate. The solvent was removed on a rotary evaporator and the residue was dissolved in 20 ml of chloroform and 0.5 ml of triethyl amine and 1 ml of acetic chloride were added and the mixture was stirred for 10 mins. Methanol was added to quench the reaction. The reaction mixture was washed with water, 2 M sodium carbonate, and brine. The organic phase was dried with sodium sulfate. After the solvent was removed on a rotary evaporator, the residue was purified by column eluted with hexane/CH₂Cl₂ (2:1) to offer 0.14 g of 4-(p-acetylthiophenyl)-2,2′,5,5′-tetramethoxy-biphenyl (yield 66%). ¹HNMR (CDCl₃) δ 7.684 (d, 2H, J=8.0 Hz), 7.511 (d, 2H, J=8.0 Hz), 7.009-6.917 (m, 5H), 3.840-3.789 (m, 12H), 2.483 (s, 3H), 3.825 (s, 3H), 3.785 (s, 3H), 2.484 (s, 1H). ¹³CNMR (CDCl₃) δ194.761, 153.804, 151.728, 151.565, 150.631, 140.238, 134.385, 130.778, 129.692, 128.739, 128.237, 126.719, 117.718, 115.633, 114.875, 113.884, 112.905, 57.014, 56.948, 56.671, 56.187, 30.647.

### Example 24

### Preparation of acetylthiophenyl-4-2′,5′-dimethoxyphenyl-tetrafluorobenzene (24)

In a 50 ml two neck round-bottom flask, 0.225 g (0.5 mmol) tert-butylthiophenyl-4-2′,5′-dimethoxyphenyl-tetrafluorobenzene was dissolved in 20 ml of chloroform and then a solution of 0.4 g (1 mmol) mercury(II) perchlorate hydrate in 10 ml of methanol was added drop by drop and yellow solids came out when enough mercury salts were added and the system was stirred overnight under room temperature. Then H₂S was bubbled into the reaction system with argon slowly until all the yellow solids turned into dark. The solids were filtered through Celite. The filtrate was washed with brine and dried with sodium sulfate. After the solvent was removed on a rotary evaporator, the residue was dissolved in 20 ml of chloroform and 0.5 ml of triethyl amine and 1 ml of acetic chloride were added. After stirred for 10 mins, 3 ml of methanol was added to quench the reaction. The mixture was washed with water, 2 M sodium carbonate, and brine and then dried with sodium sulfate. The solvent was removed on a rotary evaporator, the residue was purified by column eluted with hexane/CH₂Cl₂ (3:1) to offer 0.11 g of acetylthiophenyl-4-2′,5′-dimethoxyphenyl-tetrafluorobenzene (yield 50%). ¹HNMR (CDCl₃) δ 7.590 (s, 4H), 7.021 (d, 2H), 6.896 (s, 1H), 3.836 (s, 6H), 2.495 (s, 3H). ¹³CNMR (CDCl₃) δ193.770, 153.803, 151.821, 134.716, 131.339, 129.543, 129.244, 117.588, 117.430, 116.383, 112.869, 56.764, 56.269, 30.754.

### Example 25

### Preparation of 4-pentafluorophenyl-4′-acetylthio-2,5-dimethoxybiphenyl (25)

In a 50 ml two neck round-bottom flask, 0.23 g (0.49 mmol) 4-pentafluorophenyl-4′-(tert-butylthio)-2,5-dimethoxybiphenyl was dissolved in 20 ml of chloroform and then a solution of 0.4 g (1 mmol) mercury(II) perchlorate hydrate in 10 ml of methanol was added drop by drop and yellow solids came out when enough mercury salts were added. After stirred overnight under room temperature, H₂S was bubbled into the reaction system with argon slowly until all the yellow solids turned into dark. The solids were filtered through Celite. The filtrate was washed with brine and dried with sodium sulfate. The solvent was removed on a rotary evaporator and the residue was dissolved in 20 ml of chloroform and 0.5 ml of triethyl amine and 1 ml of acetic chloride were added. The mixture was stirred for 10 mins. Methanol was added to quench the reaction. The reaction mixture was washed with water, 2 M sodium carbonate, and brine. The organic phase was dried with sodium sulfate. After the solvent was removed on a rotary evaporator, the residue was purified by column eluted with hexane/CH₂Cl₂ (3:1) to offer 0.12 g of 4-pentafluorophenyl-4′-acetylthio-2,5-dimethoxybiphenyl (yield 54%). ¹HNMR (CDCl₃) δ7.654 (d, 2H, J=8.0 Hz), 7.518 (d, 2H, J=8.0 Hz), 7.032 (s, 1H), 6.783 (s, 1H), 3.825 (s, 3H), 3.785 (s, 3H), 2.484 (s, 1H). ¹³CNMR (CDCl₃) δ194.548, 151.735, 150.730, 139.514, 134.478, 132.422, 130.685, 127.383, 115.545, 115.332, 111.669, 56.775, 30.673.

### Example 26

### Preparation of 4-(2″,5″-dimethoxyphenyl)-4′-(p-acetylthiophenyl)-2,2′,5,5′-tetramethoxybiphenyl (26)

In a 50 ml two neck round-bottom flask, 0.17 g (0.3 mmol) 4-(2″,5″-dimethoxyphenyl)-4′-(p-tert-butylthiophenyl)-2,2′,5,5′-tetramethoxybiphenyl was dissolved in 20 ml of chloroform and then a solution of 0.4 g (1 mmol) mercury(II) perchlorate hydrate in 10 ml of methanol was added into the system drop by drop and yellow solids came out when enough mercury salt was added. Kept the system stirring overnight under room temperature. Then H₂S was bubbled into the reaction system with argon slowly until all the yellow solids turned into dark. The solids were filtered through Celite. The filtrate was washed with brine and dried with sodium sulfate. The solvent was removed on a rotary evaporator, and the residue was dissolved in 20 ml of chloroform and 0.5 ml of triethyl amine and 1 ml of acetic chloride were added. The mixture was stirred for 10 mins then 3 ml of methanol was added to quench the reaction. The reaction mixture was washed with water, 2 M sodium carbonate, and brine and then dried over sodium sulfate. After the solvent was removed on a rotary evaporator, the residue was purified by column eluted with hexane/CH₂Cl₂ (1:3) to offer 79 mg of 4-(2″,5″-dimethoxyphenyl)-4′-(p-acetylthiophenyl)-2,2′,5,5′-tetramethoxybiphenyl (yield 47%). ¹HNMR (CDCl₃) δ 7.685 (d, 2H, J=8.0 Hz), 7.508 (s, 2H, J=8.0 Hz), 7.044-6.923 (m, 7H), 3.843-3.787 (m, 18H), 2.482 (s, 3H). ¹³CNMR (CDCl₃) δ194.778, 153.783, 151.769, 151.613, 151.171, 151.127, 150.633, 140.254, 134.396, 130.789, 129.647, 129.031, 128.293, 127.866, 127.321, 126.713, 117.769, 115.871, 115.578, 115.517, 114.888, 113.840, 112.888, 57.037, 56.955, 56.923, 56.716, 56.138, 30.654.

### Example 27

### Preparation of 1-tert-acetylthiophenyl-4-2′,2″,5′,5″-tetramethoxybiphenyl-tetrafluorobenzene (27)

In a 50 ml two neck round-bottom flask, 0.18 g (0.3 mmol) 1-tert-butylthiophenyl-4-2′,2″,5′,5″-tetramethoxybiphenyl-tetrafluorobenzene was dissolved in 20 ml of chloroform and then a solution of 0.4 g (1 mmol) mercury(II) perchlorate hydrate in 10 ml of methanol was added into the system drop by drop and yellow solids came out when enough mercury salts were added. The reaction mixture was stirred overnight under room temperature. Then H₂S was bubbled into the reaction system with argon slowly until all the yellow solids turned into dark. The solids were filtered through Celite. The filtrate was washed with brine and dried with sodium sulfate. The solvent was removed on a rotary evaporator, and the residue was dissolved in 20 ml of chloroform and 0.5 ml of triethyl amine and 1 ml of acetic chloride were added and the mixture was stirred for 10 mins. Methanol was added to quench the reaction. The organic phase was washed with water, 2 M sodium carbonate, and brine and then dried over sodium sulfate. After the solvent was removed on a rotary evaporator, the residue was purified by column eluted with hexane/CH₂Cl₂ (1:2) to offer 86 mg of 1-tert-acetylthiophenyl-4-2′,2″,5′,5″-tetramethoxybiphenyl-tetrafluorobenzene (yield 50%). ¹HNMR (CDCl₃) δ 7.601 (s, 4H), 7.011-6.944 (m, 5H), 3.839 (s, 6H), 3.813 (s, 3H), 3.787 (s, 3H), 2.501 (s, 3H). ¹³CNMR (CDCl₃) δ193.78, 153.811, 151.668, 151.368, 134.727, 131.352, 130.449, 129.531, 129.292, 128.496, 117.598, 115.486, 115.347, 114.166, 112.973, 57.005, 56.928, 56.796, 56.205, 30.795.

### Example 28

### Preparation of 4-(2′,5′-dimethoxy-4′-acetylthiophenyl)phenyl-nonafluorobiphenyl (28)

In a 50 ml two neck round-bottom flask, 0.18 g (0.3 mmol) 4-(2′,5′-dimethoxy-4′-tert-butylthiophenyl)phenyl-nonafluorobiphenyl was dissolved in 20 ml of chloroform and then a solution of 0.4 g (1 mmol) mercury(II) perchlorate hydrate in 10 ml of methanol was added drop by drop and yellow solids came out when enough mercury salts were added and the system was stirred overnight under room temperature. Then H₂S was bubbled into the reaction system with argon slowly until all the yellow solids turned into dark. The solids were filtered through Celite. The filtrate was washed with brine and dried with sodium sulfate. After the solvent was removed on a rotary evaporator, the residue was dissolved in 20 ml of chloroform and 0.5 ml of triethyl amine and 1 ml of acetic chloride were added. After stirred for 10 mins, 3 ml of methanol was added to quench the reaction. The reaction mixture was washed with water, 2 M sodium carbonate, and brine and then dried over sodium sulfate. After the solvent was removed on a rotary evaporator, the residue was purified by column eluted with hexane:CH₂Cl₂ (1:2) to offer 101 mg of 4-(2′,5′-dimethoxy-4′-acetylthiophenyl)phenyl-nonafluorobiphenyl (yield 57%). ¹HNMR (CDCl₃) δ 7.674 (d, 2H, J=8.0 Hz), 7.654 (s, 2H, J=8.0 Hz), 7.072 (s, 1H), 6.957 (s, 1H), 3.874 (s, 3H), 3.811 (s, 3H), 2.490 (s, 3H). ¹³CNMR (CDCl₃) δ194.529, 151.684, 150.770, 139.479, 134.489, 132.707, 130.698, 127.449, 115.307, 114.762, 56.830, 30.680.

### Example 29

### Preparation of 4′-acetylthio-biphenyl-4-yl-nonafluorobiphenyl (29)

In a 50 ml two neck round-bottom flask, 0.17 g (0.3 mmol) 4′-tert-butylthio-biphenyl-4-yl-nonafluorobiphenyl was dissolved in 20 ml of chloroform and then a solution of 0.4 g (1 mmol) mercury (II) perchlorate hydrate in 10 ml of methanol was added dropwise and yellow solids came out when enough mercury salts were added. The mixture was stirred overnight under room temperature. Then H₂S was bubbled into the reaction system with argon slowly until all the yellow solids turned into dark. The solids were filtered through Celite. The filtrate was washed with brine and dried with sodium sulfate. The solvent was removed on a rotary evaporator, the residue was dissolved in 20 ml of chloroform and 0.5 ml of triethyl amine and 1 ml of acetic chloride were added in the system and stirred for 10 mins. Methanol was added to quench the reaction. The reaction mixture was washed with water, 2 M sodium carbonate, and brine and then dried with sodium sulfate. After the solvent was removed on a rotary evaporator, the residue was purified by column eluted with the hexane:CH₂Cl₂ (3:1) to offer 68 mg of 4′-acetylthio-biphenyl-4-yl-nonafluorobiphenyl (yield 42%). ¹HNMR (CDCl₃) δ7.793 (d, 2H, J=8.0 Hz), 7.732 (s, 2H, J=8.0 Hz), 7.656 (d, 2H, J=8.0 Hz), 7.562 (s, 2 H, J=8.0 Hz), 2.492 (s, 3H). ¹³CNMR (CDCl₃) δ194.377, 141.857, 141.643, 135.313, 131.039, 128.369, 128.020, 127.914, 126.605, 30.693.

### Example 30

### Preparation of ethyl 4-(p-tert-butylthiophenyl)-benzoate (30)

In an argon flushed two neck round-bottom flask, a mixture of 1.46 g (5.0 mmol) of 4-tert-butylthiophenyl-4,4,5,5-tetramethyl-1,3,2-dioxaborolane, 1.25 g (5.5 mmol) of ethyl 4-bromobenzoate, 90 mg (1.5 mol %) of tetrakis(triphenylphosphine)palladium, 20 ml of 2 M sodium carbonate and 50 ml of toluene was heated at reflux overnight. After cooling down, it was extracted with ethyl acetate twice and washed with brine and dried over magnesium sulfate. The solvent was removed on a rotary evaporator, the residue was purified by column eluted with hexane/CH₂Cl₂ (5:1) to offer 1.34 g of ethyl 4-(p-tert-butylthiophenyl)-benzoate (yield 85%). ¹HNMR (CDCl₃) δ8.131 (d, 2H, J=8.0 Hz), 7.679 (d, 2H, J=8.0 Hz), 7.636 (d, 2H, J=8.0 Hz), 7.596 (d, 2H, J=8.0 Hz), 4.434 (q, 2H), 1.437 (t, 3H), 1.329 (s, 9H).

### Example 31

### Preparation of 4-tert-butylthiol-4′-hydroxymethyl-biphenyl (31)

1.30 g of ethyl 4-(p-tert-butylthiophenyl)benzoate (4.14 mmol) in 10 ml of THF was dropped into 50 ml of THF solution with 0.2 g of lithium aluminum hydride (5.2 mmol) under a ice-water bath. After stirring for two hours, 10 ml of saturated sodium sulfate solution was dropped into the system under ice-water bath very slowly until no gas was generated. After filtration, the solution was extracted with ethyl acetate (20 ml×3) and the organic layer was washed with water and brine and dried with sodium sulfate. The solvent was removed on a rotary evaporator to offer 1.0 g of 4-tert-butylthiol-4′-hydroxymethyl-biphenyl (yield 90%) and the product was used for further synthesis without further purification.

### Example 32

### Preparation of 4-tert-butylthio-4′-bromomethyl-biphenyl (32)

0.5 g of Phosphorous tribromide in 10 ml of chloroform was dropped into a 100 ml round-bottom flask with a solution of 1.0 g of 4-tert-butylthio-4′-hydroxymethyl-biphenyl (3.68 mmol) in 50 ml of chloroform at room temperature. After stirring for 1 hour, 2 ml of methanol was added to quench the reaction. The mixture was washed with water and brine, and then dried with sodium sulfate. After removing the solvent, the residue was purified with a flash column using hexane:CH₂Cl₂ (1:1) as eluent to offer 0.95 g pale yellow solid product of 4-tert-butylthio-4′-bromomethyl-biphenyl (yield 77%). ¹HNMR (CDCl₃) δ7.634-7.553 (m, 6H), 7.508 (d, 2H, J=8.0 Hz), 4.574 (s, 2 H), 1.349 (s, 9H).

### Example 33

### Preparation of p-bromophenyl-nonafluorobiphenyl-4-yl ether (33)

In a 50 ml round-bottom flask, 5 ml of 1.0 M t-BuOK (5 mmol) was dropwise added to the solution of 0.865 g of p-bromophenol in 30 ml of THF. The solution was stirred for 1 hour and then transferred to a 100 ml RBF with a solution of 2.0 g of decafluorobiphenyl (6 mmol) in 30 ml of THF in a dry ice-acetone bath quickly. Raised the temperature to room temperature and stirred for another 10 mins, then 15 ml of 2 M HCl was added to quenched the reaction. The mixture was extracted with ethyl acetate (15 ml×3) and washed with brine. After dried over sodium sulfate, the solvent was removed on a rotary evaporator, the residue was purified by recrystallization from heptane to afford 2.20 g of p-bromophenyl-nonafluorobiphenyl-4-yl ether (yield 90%). ¹HNMR (CDCl₃) δ7.506 (d, 2H), 7.353 (d, 2H).

### Example 34

### Preparation of 4′-tert-butylthiobiphenyl-nonafluorobiphenyl-4-yl methane (34)

In an argon flushed 25 ml round-bottom flask, a solution of 0.67 g of 4-tert-butylthio-4′-bromomethyl-biphenyl (2 mmol) in 10 ml of THF was stirred in a dry ice-acetone bath until the temperature was dropped to −78° C., then 1.6 ml of butyl lithium (1.25 M in hexane) was injected slowly to keep the temperature below −30° C. The reaction mixture was maintained at −50° C. for 1 hour, then raised the temperature to 0° C. and stirred for another 10 mins, then cooled the system back to −78° C. again and 1.0 g of decafluorobiphenyl (3 mmol) in 5 ml of THF was injected in one portion. Raised the temperature to room temperature slowly and stirred overnight. 10 ml of saturated sodium bicarbonate solution was added to quench the reaction and the mixture was extracted with ethyl acetate (5 ml×3). The organic phase was washed with brine and dried with magnesium sulfate. The solvent was removed on a rotary evaporator, the residue was purified by column eluted with hexane to offer 0.81 g of 4′-tert-butylthiobiphenyl-nonafluorobiphenyl-4-yl methane (yield 70%). ¹HNMR (CDCl₃) δ7.621-7.550 (m, 6H), 7.314 (d, 2H), 3.031 (s, 2H), 1.346 (s, 9H). ¹³CNMR (CDCl₃) δ141.704, 141.561, 138.436, 138.200, 131.839, 129.404, 127.424, 127.326, 46.443, 37.861, 31.397.

### Example 35

### Preparation of 4′-tert-butylthiobiphenyl-4-yl-nonafluorobiphenyl-4-yl ether (35)

In an argon flushed two neck round-bottom flask, a mixture of 1.46 g (5.0 mmol) of 4-tert-butylthiophenyl-4,4,5,5-tetramethyl-1,3,2-dioxaborolane, 0.97 g of p-bromophenyl-nonafluorobiphenyl-4-yl ether, (2 mmol), 35 mg (1.5 mol %) of tetrakis(triphenylphosphine)palladium, 20 ml of 2 M sodium carbonate and 50 ml of toluene was heated at reflux overnight. After cooling down, it was extracted with ethyl acetate and washed with brine and dried over magnesium sulfate. The solvent was removed on a rotary evaporator, the residue was purified by column eluted with hexane/CH₂Cl₂ (8:1) to offer 0.74 g of 4′-tert-butylthiobiphenyl-4-yl-nonafluorobiphenyl-4-yl ether (yield 66%). ¹HNMR (CDCl₃) δ7.634-7.613 (m, 4H), 7.554 (d, 2H), 7.164 (d, 2H), 1.350 (s, 9H). ¹³CNMR (CDCl₃) δ156.984, 140.739, 138.279, 137.000, 132.281, 128.999, 127.320, 116.652, 46.418, 31.392.

### Example 36

### Preparation of 4′-tert-butylthiobiphenyl-4-yl-nonafluorobiphenyl-4-yl sulfide (36)

In an argon flushed 25 ml round-bottom flask, a solution of 0.32 g of tert-butylthio-4′-bromo biphenyl (1 mmol) in 10 ml of THF was stirred in a dry ice-acetone bath until the temperature was dropped to −78° C., then 0.8 ml of butyl lithium (1.25 M in hexane) was injected slowly to keep the temperature below −30° C. The reaction mixture was maintained at −50° C. for 1 hour, then raised the temperature to 0° C. and stirred for another 10 mins, then cooled the system back to −78° C. again. In another 10 ml argon flushed round-bottom flask, a solution of 0.05 g S in 5 ml of THF was cooled to −50° C. and transferred to previous RBF slowly. Keep the temperature at −50° C. for another 5 mins. Then, it was transferred to a solution of 1.0 g of decafluorobiphenyl (3 mmol) in 20 ml of THF under −50° C. and stirred for another 20 mins. Saturated sodium bicarbonate solution (10 ml) was added to quench the reaction. After extraction with ethyl acetate (10 ml×3), the organic phase was washed with brine and dried with magnesium sulfate. The solvent was removed on a rotary evaporator, the residue was purified by column eluted with hexane to offer 0.13 g of 4′-tert-butylthiobiphenyl-4-yl-nonafluorobiphenyl-4-yl sulfide (yield 22%). ¹HNMR (CDCl₃) δ7.631-7.537 (m, 8H), 1.340 (s, 9H). ¹³CNMR (CDCl₃) δ141.012, 140.500, 138.285, 132.919, 132.304, 131.675, 128.489, 127.389, 46.600, 31.397.

### Example 37

### Preparation of 4′-acetyllthio-biphenyl-nonafluorobiphenyl-4-yl methane (37)

In a 50 ml two neck round-bottom flask, 0.17 g (0.3 mmol) 4′-tert-butylthio-biphenyl-nonafluorobiphenyl-4-yl methane was dissolved in 20 ml of chloroform and then a solution of 0.4 g (1 mmol) mercury (II) perchlorate hydrate in 10 ml of methanol was added dropwise and yellow solids came out when enough mercury salts were added. The system was stirred overnight under room temperature. Then H₂S was bubbled into the reaction system with argon slowly until all the yellow solids turned into dark. The solids were filtered through Celite. The filtrate was washed with brine and dried with sodium sulfate. The solvent was removed on a rotary evaporator, the residue was dissolved in 20 ml of chloroform and 0.5 ml of triethyl amine and 1 ml of acetic chloride were added in the system and stirred for 10 mins. Methanol was added to quench the reaction. The reaction mixture was washed with water, 2 M sodium carbonate, and brine and then dried with sodium sulfate. After the solvent was removed on a rotary evaporator, the residue was purified by column eluted with the hexane/CH₂Cl₂ (2:1) to offer 102 mg of 4′-acetylthio-biphenyl-nonafluorobiphenyl-4-yl methane (yield 62%). ¹HNMR (CDCl₃) δ7.665 (d, 2H, J=8.0 Hz), 7.566 (s, 2H, J=8.0 Hz), 7.505 (d, 2H, J=8.0 Hz), 7.319 (s, 2H, J=8.0 Hz), 3.032 (s, 2H), 2.472 (s, 3H). ¹³CNMR (CDCl₃) δ194.647, 142.629, 141.733, 138.267, 135.174, 129.423, 128.170, 127.555, 126.888, 37.852, 30.633.

### Example 38

### Preparation of 4′-acetylthiobiphenyl-4-yl-nonafluorobiphenyl-4-yl ether (38)

In a 50 ml two neck round-bottom flask, 0.17 g (0.3 mmol) 4′-tert-butylthiobiphenyl-4-yl-nonafluorobiphenyl-4-yl ether was dissolved in 20 ml of chloroform and then a solution of 0.4 g (1 mmol) mercury (II) perchlorate hydrate in 10 ml of methanol was added drop by drop and yellow solids came out when enough mercury salts were added. The mixture was stirred overnight under room temperature. Then H₂S was bubbled into the reaction system with argon slowly until all the yellow solids turned into dark. The solids were filtered through Celite. The filtrate was washed with brine and dried with sodium sulfate. The solvent was removed on a rotary evaporator, the residue was dissolve in 20 ml of chloroform and 0.5 ml of triethyl amine and 1 ml of acetic chloride were added in the system and stirred for 10 mins. Methanol was added to quench the reaction. The reaction mixture was washed with water, 2 M sodium carbonate, and brine and then dried with sodium sulfate. After the solvent was removed on a rotary evaporator, the residue was purified by column eluted with the hexane/CH₂Cl₂ (2:1) to offer 110 mg of 4′-acetylthiobiphenyl-4-yl-nonafluorobiphenyl-4-yl ether (yield 67%). ¹HNMR (CDCl₃) δ7.626 (d, 4H), 7.513 (d, 2H, J=8.0 Hz), 7.162 (d, 2H), 2.477 (s, 3 H). ¹³CNMR (CDCl₃) δ194.486, 157.103, 141.644, 136.787, 135.268, 129.141, 128.152, 127.032, 116.659, 30.647.

### Example 39

### Preparation of 4′-acetylthiobiphenyl-4-yl-nonafluorobiphenyl-4-yl sulfide (39)

In a 50 ml two neck round-bottom flask, 0.12 g (0.2 mmol) 4′-tert-butylthiobiphenyl-4-yl-nonafluorobiphenyl-4-yl sulfide was dissolved in 20 ml of chloroform and then a solution of 0.4 g (1 mmol) mercury (II) perchlorate hydrate in 10 ml of methanol was added drop by drop and yellow solids came out when enough mercury salts were added. The mixture was stirred overnight under room temperature. Then H₂S was bubbled into the reaction system with argon slowly until all the yellow solids turned into dark. The solids were filtered through Celite. The filtrate was washed with brine and dried with sodium sulfate. The solvent was removed on a rotary evaporator, and the residue was dissolved in 20 ml of chloroform and 0.5 ml of triethyl amine and 1 ml of acetic chloride were added. The reaction mixture was stirred for 10 mins. Methanol was added to quench the reaction. The organic phase was washed with water, 2 M sodium carbonate, and brine and dried over sodium sulfate. After the solvent was removed on a rotary evaporator, the residue was purified by column eluted with hexane/CH₂Cl₂ (2:1) to offer 87 mg of 4′-acetylthiobiphenyl-4-yl-nonafluorobiphenyl-4-yl sulfide (yield 80%). ¹HNMR (CDCl₃) δ7.633-7.284 (m, 8H), 2.490 (s, 3H). ¹³CNMR CDCl₃) δ194.359, 141.417, 140.760, 135.280, 132.188, 132.007, 128.608, 128.212, 127.879, 30.674.

### Example 40

### Measurement of electronic property of 4-pentafluorophenyl-4′-acetylthio-2,5-dimethoxybiphenyl (compound 25) self assembled on Au surface

Compound 25 was dissolved in THF to a concentration of about 1 mM and concentrated ammonia was added before SAM. The molecules were assembled onto Au coated tungsten tip by immersing the tip in the solution for 21 hours. Then the tip was rinsed with THF and ethanol immediately. After that, the tip was mounted onto tip carrier before it was introduced into the UHV-STM analysis chamber to take Scanning Tunneling Spectroscopy (STS) measurement. Instead of self-assembling the target molecules on Au substrate, we use this new approach to self-assembly the molecules on Au coated tungsten tip, which is able to afford more reliable and reproducible I-V measurement results. The approach for the I-V measurement is shown in FIG. 7. Diode characteristics of compound 25 in the configuration of Au substrate/compound 25 self-assembled on Au-coated tungsten tip has been demonstrated when a positive bias applied on tip side and Au substrate grounded. The rectification ratio is about 3 at 2 V. The I-V curve is shown in FIG. 8.

### Example 41

Comparison of the redox behaviour of three conjugated molecules, namely 4-(p-tert-Butylthiophenyl)-2,2′,5,5′-tetramethoxybiphenyl (TSBOO), p-tert-Butylthiophenyl-nonafluorobiphenyl (TSBFF), tert-Butylthiophenyl-4-(2′,5′-dimethoxyphenyl)-tetrafluorobenzene (TSBFO) was investigated by cyclic voltammetry. The conjugated molecules were dissolved and scanned positively in 0.10 M Bu₄NPF₆ in anhydrous acetonitrile and anodic scan was performed. The cyclic voltammograms of the conjugated molecules are illustrated in FIG. 10. Among the molecules, 4-(p-tert-Butylthiophenyl)-2,2′,5,5′-tetramethoxybiphenyl (TSBOO), a pure p-type conjugated molecule, shows three irreversible oxidation peaks at 1.23 V, 1.41 V, and 1.73 V vs SCE. p-tert-Butylthiophenyl-nonafluorobiphenyl (TSBFF), a pure n-type molecule, demonstrates two irreversible oxidation peaks with higher oxidation potentials at 1.70 V and 1.90 V vs SCE. tert-Butylthiophenyl-4-(2′,5′-dimethoxyphenyl)-tetrafluorobenzene (TSBFO), a p-n diblock molecule, shows a reversible oxidation/reduction peak pair at 1.47/1.36 V vs SCE. The CV measurements indicate that the redox behaviour of the conjugated molecules can be largely manipulated by incorporation of different segments with different electronic properties. The redox behavior is directly relevant to the charge transport process in that the easier the oxidation of the molecule, the easier it is move an electron along the molecule.

As can be understood by one skilled in the art, many modifications to the exemplary embodiments described herein are possible. The invention, rather, is intended to encompass all such modification within its scope, as defined by the claims.

1. A conjugated molecule comprising:
from 3 to 100 Ar groups forming the backbone of the molecule, each Ar group being an arylene, an arylene-vinylene or an arylene-ethynylene group, at least one of the Ar groups being substituted with one or more electron-donating groups to form a p-type Ar group and at least one of the Ar groups being substituted with one or more electron-withdrawing groups to form an n-type Ar group, the p-type Ar group being adjacent to the n-type Ar group to form a p/n junction;
an alligator clip group (AC group) at one end of the backbone, the AC group capable of reacting with a conducting surface, wherein the AC group is acetylthio, methylthio, tert-butylthio, benzylthio, isocyano, diazo, phosphate, phosphonio, or phosphonitryl, any of which acetylthio, methylthio, tert-butylthio, benzylthio, phosphate or phosphonio is optionally substituted with one or more of C₁₋₁₈ alkyl, C₁₋₁₈ alkenyl, C₁₋₁₈ alkynyl, amido, carbonyl, sulfonyl, thioamide, C₅₋₃₀ aryl, C₅₋₃₀ arylamino, amino, nitro, cyano, isocyano or halide; and
one or more spacer groups, each spacer group independently inserted between two Ar groups, the one or more spacer groups independently selected from methylene, ethylene, propenylene, ethylene dioxy, 1,4-cyclohexylene, 1,4-cyclohexylene dioxy, thio, dithio, thionyl, sulfonyl, imino, carbonyl, carbonyl dioxy, thiocarbonyl, phosphinidene and phosphonitryl, any of which methylene, ethylene, propenylene, ethylene dioxy, 1,4-cyclohexylene, 1,4-cyclohexylene dioxy is optionally substituted with one or more C₁₋₁₈ alkyl, C₁₋₁₈ alkenyl, C₁₋₁₈ alkynyl, amido, carbonyl, sulfonyl, thioamide, C₅₋₃₀ aryl, C₅₋₃₀ arylamino, amino, nitro, cyano, isocyano or halide.

2. (canceled)

3. The conjugated molecule of claim 1 comprising from 3 to 20 Ar groups.

4. The conjugated molecule of claim 1 wherein each Ar group is independently selected from phenylene, naphthylene, thienylene, furylene, pyrrolylene, pyridylene, thiazolylene, oxadiazolylene, pyrazinylene, fluorenylene, indenofluorenylene, carbazolylene, indenocarbazolylene, dibenzofuranylene, dibenzothienylene, anthracenylene, tetracenylene, pentacenylene, indenylene, azulenylene, pentalenylene, heptalenylene, biphenylenylene, indacenylene, acenaphthenylene, phenalenylene, phenanthrylene, triphenylenylene, pyrenylene, naphthacenylene, hexacenylene, pyrazolylene, imidazolylene, naphthothienylene, thianthrenylene, pyranylene, isobenzofuranylene, chromenylene, xanthenylene, phenoxathiinylene, pyrimidinylene, pyridazinylene, indolizinylene, isoindolylene, indolylene, purinylene, quinolizinylene, quinolylene, phthalazinylene, pteridinylene, acridinylene, phenanthridinylene, pyrrolinylene, imidazolinylene, indolinylene, phenylene vinylene, naphthylene vinylene, thienylene vinylene, furylene vinylene, pyrrolylene vinylene, pyridylene vinylene, thiazolylene vinylene, oxadiazolylene vinylene, pyrazinylene vinylene, fluorenylene vinylene, indenofluorenylene vinylene, carbazolylene vinylene, indenocarbazolylene vinylene, dibenzofuranylene vinylene, dibenzothienylene vinylene, anthracenylene vinylene, tetracenylene vinylene, pentacenylene vinylene, indenylene vinylene, azulenylene vinylene, pentalenylene vinylene, heptalenylene vinylene, biphenylenylene vinylene, indacenylene vinylene, acenaphthenylene vinylene, phenalenylene vinylene, phenanthrylene vinylene, triphenylenylene vinylene, pyrenylene vinylene, naphthacenylene vinylene, hexacenylene vinylene, pyrazolylene vinylene, imidazolylene vinylene, naphthothienylene vinylene, thianthrenylene vinylene, pyranylene vinylene, isobenzofuranylene vinylene, chromenylene vinylene, xanthenylene vinylene, phenoxathiinylene vinylene, pyrimidinylene vinylene, pyridazinylene vinylene, indolizinylene vinylene, isoindolylene vinylene, indolylene vinylene, purinylene vinylene, quinolizinylene vinylene, quinolylene vinylene, phthalazinylene vinylene, pteridinylene vinylene, acridinylene vinylene, phenanthridinylene vinylene, pyrrolinylene vinylene, imidazolinylene vinylene, indolinylene vinylene, phenylene ethynylene, naphthylene ethynylene, thienylene ethynylene, furylene ethynylene, pyrrolylene ethynylene, pyridylene ethynylene, thiazolylene ethynylene, oxadiazolylene ethynylene, pyrazinylene ethynylene, fluorenylene ethynylene, indenofluorenylene ethynylene, carbazolylene ethynylene, indenocarbazolylene ethynylene, dibenzofuranylene ethynylene, dibenzothienylene ethynylene, anthracenylene ethynylene, tetracenylene ethynylene, pentacenylene ethynylene, indenylene ethynylene, azulenylene ethynylene, pentalenylene ethynylene, heptalenylene ethynylene, biphenylenylene ethynylene, indacenylene ethynylene, acenaphthenylene ethynylene, phenalenylene ethynylene, phenanthrylene ethynylene, triphenylenylene ethynylene, pyrenylene ethynylene, naphthacenylene ethynylene, hexacenylene ethynylene, pyrazolylene ethynylene, imidazolylene ethynylene, naphthothienylene ethynylene, thianthrenylene ethynylene, pyranylene ethynylene, isobenzofuranylene ethynylene, chromenylene ethynylene, xanthenylene ethynylene, phenoxathiinylene ethynylene, pyrimidinylene ethynylene, pyridazinylene ethynylene, indolizinylene ethynylene, isoindolylene ethynylene, indolylene ethynylene, purinylene ethynylene, quinolizinylene ethynylene, quinolylene ethynylene, phthalazinylene ethynylene, pteridinylene ethynylene, acridinylene ethynylene, phenanthridinylene ethynylene, pyrrolinylene ethynylene, imidazolinylene ethynylene and indolinylene ethynylene.

5. The conjugated molecule of claim 1 wherein one or more Ar groups is substituted with one or more substituents independently selected from the group consisting of linear or branched C₁₋₁₈ alkyl, linear or branched C₂₋₁₈ alkenyl, linear or branched C₂₋₁₈ alkynyl, linear or branched C₁₋₁₈ alkoxy, linear or branched C₁₋₁₈ alkylamino or dialkylamino, linear or branched C₁₋₁₈ alkylthio, amido, carbonyl, carboxyl, alkyl sulfonyl, sulfo, sulfonyl, thioamide, ^{|}C₅₋₃₀ aryl, C₅₋₃₀ arylamino, C₅₋₃₀ diarylamino, amino, ammonio, hydroxyl, nitro, cyano, isocyano and halide, wherein any of the C₁₋₁₈ alkyl, C₂₋₁₈ alkenyl, C₂₋₁₈ alkynyl, C₁₋₁₈ alkoxy, C₁₋₁₈ alkylamino or dialkylamino, or C₁₋₁₈ alkylthio optionally contains one to four heteroatoms selected from the group consisting of N, O, S, Si and P, and wherein any of the C₁₋₁₈ alkyl, C₂₋₁₈ alkenyl, C₂₋₁₈ alkynyl, C₁₋₁₈ alkoxy, C₁₋₁₈ alkylamino or dialkylamino, C₁₋₁₈ alkylthio, C₅₋₃₀ aryl, C₅₋₃₀ arylamino or C₅₋₃₀ diarylamino is optionally further substituted, including with one or more halides.

6. The conjugated molecule of claim 1 wherein the one or more electron-withdrawing groups are independently selected from halide, carbonyl, carboxyl, cyano, ammonio, nitro, nitroso, sulfonyl, amido linked to the backbone through the oxygen, pyridinium, phosphonium, pyridyl, thiazolyl, oxadiazolyl and triazolyl.

7. The conjugated molecule of claim 1 wherein the one or more electron-donating groups are independently selected from alkoxyl, alkylthio, amino, hydroxyl, amido connected to the backbone through the nitrogen, carboxyl connected to the backbone through the oxygen, phenyl, naphthyl, thiophenyl, furanyl, pyrrolyl, carbazolyl, alkyl, alkenyl and alkynyl.

8. The conjugated molecule of claim 1 wherein a first segment of the Ar groups is p-type and a second segment of the Ar groups is n-type.

9. The conjugated molecule of claim 1 further comprising alternating p-type segments and n-type segments.

10. (canceled)

11. (canceled)

12. The conjugated molecule of claim 1, wherein the AC group is acetylthio.

13. A conjugated molecule that is p-tert-butylthiophenyl-nonafluorobiphenyl, tert-butylthiophenyl-4-(2′,5′-dimethoxyphenyl)-tetrafluorobenzene, 4-pentafluorophenyl-4′-tert-butylthio-2,5-dimethoxybiphenyl, 4-pentafluorophenyl-4′-(p-tert-butylthio)phenyl-2,2′,5′5′-tetramethoxybiphenyl, 1-tert-butylthiophenyl-4-(2′,2″,5′,5″-tetramethoxybiphenyl)-tetrafluorobenzene, 4-(2′,5′-dimethoxy-4′-tert-butylthiophenyl)phenyl-nonafluorobiphenyl, 4′-tert-butylthio-biphenyl-4-yl-nonafluorobiphenyl, p-acetylthiophenyl-nonafluorobiphenyl, acetylthiophenyl-4-2′,5′-dimethoxyphenyl-tetrafluorobenzene, 4-pentafluorophenyl-4′-acetylthio-2,5-dimethoxybiphenyl, 1-tert-acetylthiophenyl-4-2′,2″,5′,5″-tetramethoxybiphenyl-tetrafluorobenzene, 4-(2′,5′-dimethoxy-4′-acetylthiophenyl)phenyl-nonafluorobiphenyl, 4′-acetylthio-biphenyl-4-yl-nonafluorobiphenyl, 4′-tert-butylthio-biphenyl-nonafluorobiphenyl-4-yl methane, 4′-tert-butylthio-biphenyl-4-yl-nonafluorobiphenyl-4-yl ether, 4′-tert-butylthio-biphenyl-4-yl-nonafluorobiphenyl-4-yl sulfide, 4′-acetyllthio-biphenyl-nonafluorobiphenyl-4-yl methane, 4′-acetylthio-biphenyl-4-yl-nonafluorobiphenyl-4-yl ether or 4′-acetylthio-biphenyl-4-yl-nonafluorobiphenyl-4-yl sulfide.

14. A molecular electronic device, comprising a first electrical contact, a second electrical contact, and the conjugated molecule of claim 1 forming a conductive path between the first electrical contact and the second electrical contact, wherein the second electrical contact is connected to the conjugated molecule through the AC group.

15. The molecular electronic device of claim 14 wherein the first electrical contact and the second electrical contact are each independently composed of gold, silver, copper, platinum, palladium, indium tin oxide or a conductive polymer.

16. The molecular electronic device of claim 14 wherein the first electrical contact and the second electrical contact are each layered on a solid substrate.

17. The molecular electronic device of claim 14 wherein the solid substrate is silicon, mica, polyethylene terephthalate or polycarbonate.

18. The molecular electronic device of claim 14 wherein the conjugated molecule is a plurality of conjugated molecules.

19. The molecular electronic device of claim 18 wherein the plurality of conjugated molecules forms a monolayer.

20. A method of manufacturing the molecular electronic device of claim 14, comprising contacting the first electrical contact with a solution containing the conjugated molecule of claim 1 to form a monolayer of conjugated molecule on the first electrical contact, the first electrical contact contacting the AC group of the conjugated molecule; and depositing a second electrical contact on the monolayer of conjugated molecule, the second electrical contact contacting the end of the conjugated molecule not having the AC group.

21. A crossbar device comprising a first conductor and a second conductor that intersects the first conductor at a non-zero angle and the molecular electronic device of claim 14, wherein the molecular electronic device connects the first conductor and the second conductor at the point of intersection.
